SEQUENCE LISTING (2 sequences)

SEQ ID NO: 1 - DNA, 20 bp, Artificial
  synthetic primer
tggagttccccagattgaag

SEQ ID NO: 2 - DNA, 20 bp, Artificial
  synthetic primer
cctgacccacctttttctca

(19) United States
(12) Patent Application Publication
(10) Pub. No.: US 2010/0075894 A1
(43) Pub. Date: Mar. 25, 2010

(54) REDUCING ER STRESS IN THE TREATMENT OF OBESITY AND DIABETES

(75) Inventors: Gökhan S. Hotamisligil, Wellesley, MA (US); Umut Özcan, Brookline, MA (US)

Correspondence Address: EDWARDS ANGELL PALMER & DODGE LLP, P.O. BOX 55874, BOSTON, MA, 02205, US

(73) Assignee: HARVARD UNIVERSITY, Cambridge, MA (US)

(21) Appl. No.: 12/541,020

(22) Filed: Aug. 13, 2009

Related U.S. Application Data
(63) Continuation of application No. 11/227,497, filed on Sep. 15, 2005, now abandoned.
(60) Provisional application No. 60/610,093, filed on Sep. 15, 2004.

(51) Int. Cl. A61K 38/28; G01N 33/53; A61K 31/192; A61K 31/575; A61K 31/04; A61K 31/155; A61K 31/60; A61P 3/10; A61P 3/04; A61P 9/10

(52) U.S. Cl. 514/4; 435/7.2; 436/86; 514/570; 514/169; 514/740; 514/171; 514/635; 514/165

(57) ABSTRACT

Endoplasmic reticulum stress has been found to be associated with obesity. Therefore, agents that reduce or prevent ER stress may be used to treat diseases associated with obesity including peripheral insulin resistance, hypergylcemia, and type 2 diabetes. Two compounds which have been shown to reduce ER stress and to reduce blood glucose levels include 4-phenyl butyric acid (PBA), tauroursodeoxycholic acid (TUDCA), and trimethylamine N-oxide (TMAO). Other compounds useful in reducing ER stress are chemical chaperones such as trimethylamine N-oxide and glycerol. The present invention provides methods of treating a subject suffering from obesity, hyperglycemia, type 2 diabetes, or insulin resistance using ER stress reducers such as PBA, TUDCA, and TMAO. Methods of screening for ER stress reducers by identifying agents that reduce levels of ER stress markers in ER stressed cells are also provided. These agents may find use in methods and pharmaceutical compositions for treating obesity-associated diseases.

### RELATED APPLICATIONS

The present application claims priority under 35 U.S.C. §119(e) to U.S. provisional patent application, U.S. Ser. No. 60/610,093, filed Sep. 15, 2004, which is incorporated herein by reference. This application is also related to U.S. provisional patent application, U.S. Ser. No. 60/610,286, filed Sep. 15, 2004, entitled “Modulation of XBP-1 activity for treatment of metabolic disorders”; U.S. patent application, U.S. Ser. No. ______, filed Sep. 15, 2005, entitled “Modulation of XBP-1 activity for treatment of metabolic disorders”; and U.S. patent application, U.S. Ser. No. 10/655,620, filed Sep. 2, 2003, entitled “Methods and Compositions for Modulating XBP-1 Activity”, each of which is incorporated herein by reference.

### GOVERNMENT SUPPORT

The work described herein was supported, in part, by a grant no. 32412 from the National Institutes of Health. The United States government may have certain rights in the invention.

### BACKGROUND OF THE INVENTION

The dramatic increase in the incidence of obesity in most parts of the world has contributed to an increased incidence of insulin resistance, type 2 diabetes, and cardiovascular disease. These obesity-associated diseases have become serious threats to human health.

Obesity has been found to be associated with the activation of cellular stress signaling pathways (Uysal et al. Nature 389:610, 1997; Hirosumi et al. Nature 420:333, 2003; Yuan et al. Science 293:1673, 2001; each of which is incorporated herein by reference). One player in the cellular stress response is the endoplasmic reticulum (ER), a membranous network that functions in the synthesis and processing of secretory and membrane proteins. The ER is responsible for the processing and translocation of most secreted and integral membrane proteins of eukaryotic cells. The lumen of the ER provides a specialized environment for the posttranslational modification and folding of these proteins. Properly folded proteins are cleared for exit from the ER and progress down the secretory pathway, while unfolded or misfolded proteins are disposed of by ER-associated protein degradation machinery. The load of proteins that cells process varies considerably depending on the cell type and physiological state of the cell. Cells can adapt by modulating the capacity of their ER to process proteins and the load of protein synthesized. Disequilibrium between ER load and folding capacity is referred to as ER stress (Harding et al. Diabetes 51 (Supp. 3):S455, 2002; incorporated herein by reference). ER stress has been shown to be triggered by hypoxia, hypoglycemia, exposure to natural toxins that perturb ER function, and a variety of mutations that affect the ability of client proteins to fold (Lee, Trends Biochem. Sci. 26:504-510, 2001; Lee, Curr. Opin. Cell Biol. 4:267-273, 1992; each of which is incorporated herein by reference).

Certain pathological conditions have been shown to disrupt ER homeostasis thereby leading to the accumulation of unfolded and misfolded proteins in the ER lumen (Hampton Curr. Biol. 10:R518, 2000; Mori Cell 101:451, 2000; Harding et al. Annu. Rev. Cell Dev. Biol. 18:575, 2002; each of which is incorporated herein by reference). To cope with ER stress, cells activate a signal transduction system linking the ER lumen with the cytoplasm and nucleus, called the unfolded protein response (UPR) (Hampton Curr. Biol. 10:R518, 2000; Mori Cell 101:451, 2000; Harding et al. Annu. Rev. Cell Dev. Biol. 18:575, 2002; each of which is incorporated herein by reference). Among the conditions that trigger ER stress are glucose and nutrient deprivation, viral infections, increased synthesis of secretory proteins, and the expression of mutant or misfolded proteins (Ma et al. Cell 107:827, 2001; Kaufman et al. Nat. Rev. Mol. Cell. Biol. 3:411, 2002; each of which is incorporated herein by reference).

### SUMMARY OF THE INVENTION

Many of the conditions that have been shown to trigger ER stress have also been found to occur in obesity, and associated diseases such as type 2 diabetes, hyperglycemia, and insulin resistance. For example, obesity increases the demand on the synthetic machinery of the cell in many secretory organ systems and is also associated with abnormalities in intracellular energy fluxes and nutrient availability. The present invention stems from the recognition that many of these diseases associated with obesity cause ER stress, particularly in peripheral tissues, and that ER stress is involved in triggering insulin resistance and type 2 diabetes, two sequelae of obesity. Therefore, agents that reduce ER stress are useful in treating obesity, peripheral insulin resistance, hyperglycemia, and type 2 diabetes. The agents useful in the treatment of these diseases include small molecules, proteins, nucleic acids, and any other chemical compounds known to reduce or prevent ER stress. These agents make act in any manner that reduces or prevents ER stress such as reducing the production of mutant or misfolded proteins, increasing the expression of ER chaperones, increasing the stability of proteins, boosting the processing capacity of the ER, etc. Particularly useful agents include chemical chaperones such as 4-phenyl butyrate (PBA), tauroursodeoxycholic acid (TUDCA), trimethylamine N-oxide (TMAO), glycerol, D₂O, dimethylsufloxide, glycine betaine, methyl amines, and glycerophosphocholine. In particular, both PBA and TUDCA have been shown to regulate ER stress in animals as measured by the reduced phosphorylation of PERK, reduced activation of JNK, and reduced phosphorylation of IRE-1α, as determined by western blot after treatment of the animal with the compound. In addition, both PBA and TUDCA regulate insulin receptor signaling in animals, as measured by increased tyrosine phosphorylation of insulin receptor, insulin substrate 1-IRS-1 and IRS-1, and increased serine phosphorylation of Akt. TMAO has been shown to act as an anti-diabetic agent in vivo, lowering glucose and insulin levels when administered to animals with insulin resistance and type 2 diabetes. The agent or a pharmaceutical composition of the agent is administered to a subject (e.g., human, dog, cat, mammal, animal) in doses effective to reduce ER stress, and thereby reduce the signs, symptoms, and consequences of obesity, insulin resistance, and type 2 diabetes. The invention also provides methods of treating and/or preventing obesity, insulin resistance, type 2 diabetes, and hyperglycemia by administering agents that reduce ER stress. The agents may be administered in any manner known in the drug delivery art although preferably the agent is delivered orally or parenterally. Dose ranges for these agents depend on the agent being delivered as well as other factors but will typically be from 10 mg/kg/day to 10 g/kg/day.

In certain embodiments, the agent used to reduce ER stress is 4-phenyl butyric acid (PBA).

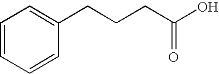

PBA has been shown to regulate ER stress and regulate insulin signaling. Phenyl butyric acid (PBA) or a derivative or salt thereof is administered to a subject in order to reduce ER stress and is particularly useful in the treatment of obesity, diabetes type 2, insulin resistance, and reducing blood glucose. PBA is effective in reducing blood glucose and increasing insulin sensitivity (FIGS. 4 and 5). PBA, or a pharmaceutical composition thereof, is administered in doses ranging from 10 mg/kg/day to 2 g/kg/day, preferably from 100 mg/kg/day to 1 g/kg/day, more preferably from 500 mg/kg/day to 1 g/kg/day.

In another embodiment, tauroursodeoxycholic acid (TUDCA), a bile acid, is the agent used to reduce ER stress.

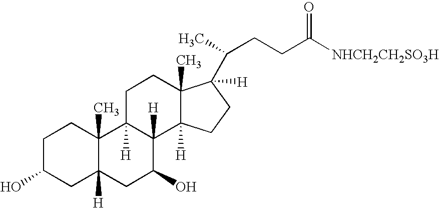

TUDCA has been shown to regulate ER stress and insulin signaling. The invention provides the administration of tauroursodeoxycholic acid (TUDCA) or a salt or derivative thereof to a subject in order to reduce ER stress. TUDCA has been found to reduce blood glucose levels and increase insulin sensitivity making it useful in the treatment of obesity, diabetes type 2, and insulin resistance (FIG. 6). TUDCA, or a pharmaceutical composition thereof, is administered in doses ranging from 10 mg/kg/day to 2 g/kg/day, preferably from 100 mg/kg/day to 1 g/kg/day, more preferably from 250 mg/kg/day to 750 mg/kg/day.

In another embodiment, TMAO is the agent used to reduce ER stress.

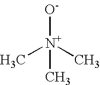

TMAO has been shown to act as an anti-diabetic agent in vivo (see FIG. 7). The invention provides the administration of TMAO or a salt or derivative thereof to a subject in order to reduce ER stress. TUDCA has been found to reduce blood glucose levels and increase insulin sensitivity making it useful in the treatment of obesity, diabetes type 2, and insulin resistance (FIG. 7). TMAO, or a pharmaceutical compositions thereof, is administered in doses ranging from 100 mg/kg/day to 0.01 g/kg/day, preferably from 10 mg/kg/day to 0.1 g/kg/day, more preferably from 5 mg/kg/day to 0.5 mg/kg/day.

Pharmaceutical compositions including agents that reduce ER stress and pharmaceutically acceptable excipients are also provided. The pharmaceutical compositions may be formulated for oral, parenteral, or transdermal delivery. The ER stress reducing agent may also be combined with other pharmaceutical agents, such as insulin, anti-diabetics, hypoglycemic agents, cholesterol lowering agents, appetite suppressants, aspirin, vitamins, minerals, and anti-hypertensive agents. For example, PBA may be combined with or administered in conjunction with metformin. The agents may be combined in the same pharmaceutical composition or may be kept separate (i.e., in two separate formulations) and provided together in a kit. The kit may also include instructions for the physician and/or patient, syringes, needles, box, bottles, vials, etc.

In another aspect, the invention provides a method of screening for agents that reduce ER stress. The identified agents are useful in the treatment of obesity, type 2 diabetes, hyperglycemia, and insulin resistance. Agents to be screened are contacted with cells experiencing ER stress. The ER stress experienced by the cells may be caused by genetic alteration or treatment with a chemical compounds known to cause ER stress (e.g., tunicamycin, thapsigargin). Cells particularly useful in the inventive screen include liver cells and adipose cells. The levels of ER stress markers are then determined to identify agents that reduce ER stress. Examples of markers of ER stress include spliced forms of XBP-1, the phosphorylation status of PERK (Thr980) and eIF2α (Ser51), mRNA and protein levels of GRP78/BIP, and JNK activity. Agents that when contacted with a cell with ER stress cause a reduction in the markers of ER stress as compared to an untreated control cell are identified as agents that reduce ER stress. A decrease in the levels of an ER stress marker are indicative of an agent that is useful in treating diseases associated with ER stress, such as obesity, type 2 diabetes, insulin resistance, hyperglycemia, cystic fibrosis, and Alzheimer's diseases. Agents identified using the inventive method are part of the invention. These agents may be further tested for use in pharmaceutical compositions.

In another aspect, the invention provides a method of diagnosing insulin resistance, hyperglycemia, or type 2 diabetes by measuring the level of expression of ER stress markers. Markers which may be analyzed in the inventive diagnostic method include spliced forms of XBP-1, phosphorylation status of PERK, phosphorylation of eIF2α, mRNA levels of GRP78/BIP, protein levels of GRP78/BIP, and JNK activity. Any other cellular marker known to be indicative of ER stress may also be used. The levels of these markers may be measured by any method known in the art including western blot, northern blot, immunoassay, or enzyme assay. An increase in the level of an ER stress markers indicates that the subject it at risk for insulin resistance, hyperglycemia, or type 2 diabetes.

### DEFINITIONS

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). In certain embodiments, the animal is a human.

“Chemical chaperone”: A “chemical chaperone” is a compound known to stabilize protein conformation against denaturation (e.g., chemical denaturation, thermal denaturation), thereby preserving protein structure and function (Welch et al. Cell Stress Chaperones 1:109-115, 1996; incorporated herein by reference). In certain embodiments, the “chemical chaperone” is a small molecule or low molecular weight compound. Preferably, the “chemical chaperone” is not a protein. Examples of “chemical chaperones include glycerol, deuterated water (D₂O), dimethylsulfoxide (DMSO), trimethylamine N-oxide (TMAO), glycine betaine (betaine), glycerolphosphocholine (GPC) (Burg et al. Am. J. Physiol. (Renal Physiol. 43):F762-F765, 1998; incorporated herein by reference), 4-phenyl butyrate or 4-phenyl butyric acid (PBA), methylamines, and tauroursodeoxycholic acid (TUDCA). Chemical chaperones may be used to influence the protein folding in a cell. Chemical chaperones have been shown in certain instances to correct folding/trafficking defects seen in such diseases as cystic fibrosis (Fischer et al. Am. J. Physiol. Lung Cell Mal. Physiol. 281:L52-L57, 2001; incorporated herein by reference), prion-associated diseases, nephrogenic diabetes insipidus, and cancer (Bai et al. Journal of Pharmacological and Toxicological Methods 40(1):3945, July 1998; incorporated herein by reference). Chemical chaperones also find use in the reduction of ER stress and are useful in the treatment of obesity, type 2 diabetes, insulin resistance, and hyperglycemia.

“Effective amount”: In general, the “effective amount” of an active agent, such as an ER stress reducer or a pharmaceutical composition thereof, refers to the amount necessary to elicit the desired biological response. As will be appreciated by those of ordinary skill in this art, the effective amount of an agent that, reduces or prevents ER stress may vary depending on such factors as the desired biological endpoint, the agent being delivered, the disease being treated, the subject being treated, etc. For example, the effective amount of agent used to treat hyperglycemia or type 2 diabetes is the amount that results in a reduction in blood glucose levels by at least about 10%, 20%, 30%, 40%, or 50%. In other embodiments, the effective amount of the ER stress modulator reduces the levels of at least one ER stress marker (e.g., spliced forms of XBP-1, phosphorylation status of PERK, phosphorylation of eIF2α, mRNA levels of GRP78/BIP, protein levels of GRP78/BIP, and JNK activity). In certain embodiments, the levels of at least two, three, four, or more ER stress markers are reduced. The ER stress marker may be reduced by approximately 10%, 20%, 30%, 40%, 50%, 60%, 70,%, 80%, 90%, 95%, 98%, 99%, or 100%.

“Peptide” or “protein”: According to the present invention, a “peptide” or “protein” comprises a string of at least three amino acids linked together by peptide bonds. The terms “protein” and “peptide” may be used interchangeably. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide” refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., fluororibose, ribose, 2′-deoxyribose, 2′-O-methylcytidine, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

### BRIEF DESCRIPTION OF THE DRAWINGS

FIG. 1 evidences increased endoplasmic reticulum stress seen in obesity. Dietary (high fat diet-induced) and genetic (ob/ob) models of mouse obesity were used to examine markers of ER stress in liver tissue compared to age and sex matched lean controls. ER stress markers including spliced forms of XBP-1 (XBP-1s), eIF2a phosphorylation (ser51, p-eIF2a), PERK phosphorylation (p-PERK), mRNA expression level of GRP78, and JNK activity were examined in the liver samples of the male mice (C57BL/6) that were kept either on standard or high fat diet for 16 weeks. 100 μg of protein were used for each immunoblot (FIG. 1a). Examination of the same ER stress markers in the livers of male, ob/ob and wild type mice, between the ages of 12-14 weeks (FIG. 1b). Expression levels of GRP78 mRNA in liver were examined by Northern blot analyses in lean and obese animals similar to the group described in FIGS. 1a and 1b (FIG. 1c).

FIG. 2 shows how the induction of ER stress impairs insulin action in liver cells via JNK-mediated phosphorylation of IRS-1. ER stress was induced in Fao cells, either with thapsigargin (thap, 300 nM for 4 hours) or tunicamycin (tun, 10 μg/ml for 2 hours), and cells were subsequently stimulated with insulin (ins). Insulin stimulated, IRS-1 tyrosine phosphorylation and total protein levels in thapsigargin- or tunicamycin-treated cells were examined after immunoprecipitation (IP) of IRS-1 followed by immunoblotting (IB) with an antibody against phospho-tyrosine (pY) (a). Insulin stimulated insulin receptor (IR) tyrosine phosphorylation and total protein levels in thapsigargin- or tunicamycin-treated cells (b). Phosphorylation of ser307 residue of IRS-1 in Fao cells after 2 hours stimulation with thapsigargin (c). Inhibition of ser307 phosphorylation by JNK-1 inhibitor SP600125 (JNKi) after thapsigargin treatment (d). Reversal of tunicamycin-induced inhibition of insulin-stimulated tyrosine phosphorylation (pY) of IRS-1 by blocking JNK activity with a peptide inhibitor (JNKi). In these experiments, both immunoprecipitation and immunoblotting were performed with anti-phosphotyrosine antibodies (e).

FIG. 3 shows how IRE-1 plays a crucial role in ER stress mediated JNK activation, ser307 phosphorylation of IRS-1, and inhibition of insulin receptor signaling. JNK activity was examined at indicated times following treatment with tunicamycin in IRE1a+/+ and IRE-1a−/− fibroblasts using an in vivo kinase assay and recombinant c-jun as substrate. JNK activity and total JNK levels (a). Phosphorylation of IRS-1 at ser307 residue in IRE1a+/+ and IRE-1a−/− fibroblasts following treatment with tunicamycin following IRS-1 immunoprecipitation (IP) and immunoblotting (IB) with an IRS-1 phosphoserine 307-specific antibody (b). Insulin (ins)-stimulated IRS-1 tyrosine phosphorylation and total IRS-1 in levels following treatment of IRE1a+/+ and IRE-1a−/− fibroblasts with tunicamycin. The graph below the blots shows the corrected density of IRS-1 tyrosine phosphorylation to total IRS-1 levels at each treatment time (c).

FIG. 4 shows how the administration of 4-phenyl butyrate (4-PBA) via parenteral route increases insulin sensitivity in vivo and lowers blood glucose levels of diabetic mice. A genetic (ob/ob) model of mouse obesity was used to analyze the effects of 4-phenyl butyrate (4-PBA) on insulin sensitivity and hyperglycemia. 10-12 weeks old, male, leptin deficient mice were obtained from Jackson Labs, acclimated by three times injection of PBS (3 times a day) for 4 days, and treated either with intraperitoneal injection of 4-PBA (1 g/kg/day in three divided doses) or phosphate buffer saline (PBS) (3 doses of 100 μl) for a period of 7 days. Fed blood glucose levels (mg/dl) at the 3^{rd} and 7^{th} days of trial (a). Insulin Tolerance Test (ITT) were performed on the 7^{th} day after the first injection (b).

FIG. 5 shows how the administration of 4-phenyl butyrate (4-PBA) via oral route increases insulin sensitivity in vivo and lowers blood glucose levels of diabetic mice. A genetic (ob/ob) model of mouse obesity was used to analyze the effects to 4-phenyl butyrate (4-PBA) on insulin sensitivity and hyperglycemia. 8-10 weeks old, male, leptin deficient mice were obtained from Jackson Labs, acclimated by PBS injection (2 times a day) for 4 days, and treated either with 4-PBA (500 mg/kg/day in two divided doses) or phosphate buffer saline (PBS) (2 doses of 200 μl) for a period of 20 days. Body weight during the treatment period (a). Blood glucose levels (mg/dl) after 6 hours of fasting at day 0 and day 20 (b). Insulin (ng/ml) levels at day 0 and day 20 (c). Insulin tolerance tests were performed at the 15^{th} day of the treatment (d).

FIG. 6 shows that the treatment of ob/ob mice with tauroursodexoycholic acid (TUDCA) increases insulin sensitivity and reverses diabetes. 9-10 weeks old ob/ob and their age and sex matched lean controls were used to analyze the effects of TUDCA on glucose metabolism and diabetes. After acclimation with PBS injection (once a day, 200 μl i.p. injection) for 4 days, mice were injected intraperitoneally with TUDCA (500 mg/kg once a day in 200 μl of PBS) (n=6 for lean and n=7 for ob/ob) and PBS (200 μl) (n=6 lean and n=7 for ob/ob). Blood glucose levels on the 4^{th}, 7^{th}, and 10^{th} days after first injection (a). ITT on 10 days after the first injection (b).

FIG. 7 shows the anti-diabetic effect of trimethylamine N-oxide (TMAO). Experiments were performed using the ob/ob genetic model of obesity and insulin resistance (C57BL/6J-Lep-ob mice purchased from Jackson Laboratory, Bar Harbor, Me.). At seven weeks of age, treatments were started after acclimatization for 5 days by administration of PBS. After this period, TMAO (Sigma, T0514) was dissolved in PBS and administered by intraperitoneal (IP) injection every 24 hours at a dose of 1 g/kg/day. At the indicated days, fed blood glucose levels were measured at 8:00 AM using an automated glucometer system. For insulin measurements, blood samples were collected after 6 hours of fasting and serum insulin levels were determined using a specific ELISA (Crystal Chem, 90060). Each treatment group contained at least six animals per experimental group.

FIG. 8 shows an effective combination of sodium 4-phenylbutyrate and metformin in the treatment of diabetes. Experiments are performed using the ob/ob genetic model of obesity and insulin resistance (C57B6. V-Lepob/OlaHsd mice were purchased from Harlan Teklad, Madison, Wis.). At seven weeks of age, treatments were started after acclimatization for 5 days by administration of PBS. After this period, PBS, sodium 4-phenylbutyrate (200 mg/kg/day), metformin (200 mg/kg/day), and sodium 4-phenylbutyrate plus metformin (200 mg/kg/day each) were administered into four separate experimental groups of mice (with at least six mice in each group) by oral gavage daily. At these doses, only the combined sodium 4-phenylbutyrate and metformin proved to be effective as a blood glucose lowering regimen and single agents did not have any effect on blood glucose. Blood glucose levels were measured at the fed state at 8:00 AM.

FIG. 9 shows regulation of GRP78 expression by glucose in vitro and hyperglycemia in vivo. (A) Fao cells were treated with various doses of glucose (0, 5, 10, 25, and 75 mM) for 24 hours. The mRNA level of GRP78 was examined by Northern blot using the total RNAs isolated from these cells. Ethidium bromide staining is shown as a control for loading and integrity of RNA. (B) Streptozotocin (STZ, 200 mg/kg) was injected intaperitoneally into male mice. Three days after injection, blood glucose levels were measured to confirm STZ-induced hyperglycemia. Livers were collected 10 days after injection and GRP78 expression was examined by Northern blot analysis using the liver total RNA.

FIG. 10 shows ER stress indicators in adipose tissues of obese mice. Dietary (high fat diet-induced) and genetic (ob/ob) models of mouse obesity were used to examine markers of ER stress in adipose tissue compared with age and sex matched lean controls. (A) PERK phosphorylation (p-PERK) and JNK activity were examined in the adipose samples of the male mice (C57BL/6) that were kept either on standard diet (RD) or high fat diet (HFD) for 16 weeks. (B) PERK phosphorylation and JNK activity in the adipose tissues of male ob/ob and WT lean mice at the ages of 12-14 weeks. (C) The mRNA levels of GRP78 were examined by Northern blot analysis in the adipose tissues of WT lean and ob/ob animals. Ethidium bromide staining is shown as a control for loading and integrity of RNA.

FIG. 11 shows the induction of ER stress impairs insulin action through JNK mediated phosphorylation of IRS-1. (A) ER stress was induced in Fao liver cells by a 3-hour treatment with 5 μg/ml tunicamycin (Tun). Cells were subsequently stimulated with insulin (Ins). IRS-1 tyrosine and serine (Ser307) phosphorylation, Akt phosphorylation (Ser473), insulin receptor (IR) tyrosine phosphorylation, and their total protein levels were examined using either immunoprecipitation (IP) followed by immunoblotting (IB) or direct immunoblotting. (B) Quantitation of IRS-1 (tyrosine and Ser307), Akt (Ser473), and IR (tyrosine) phosphorylation under the experimental conditions described in (A) with normalization to protein levels for each molecule. (C) Inhibition of ER stress-induced (300 nM thapsigargin for 4 hours) Ser307 phosphorylation of IRS-1 by JNK-1 inhibitor, SP600125 (JNKi, 25 μM), (D) Quantitation of IRS-1 Ser307 phosphorylation under conditions described in (C). (E) Reversal of ER stress-induced inhibition of insulin-stimulated tyrosine phosphorylation (pY) of IRS-1 by a JNK inhibitor. (F) Quantitation of insulin-induced IRS-1 tyrosine phosphorylation levels described in (E). (G) JNK activity, Ser307 phosphorylation of IRS-1, and total IRS-1 levels at indicated times following tunicamycin treatment (Tun, 10 μg/ml for 1 hour) in IRE-1α^{+/+} and IRE-1α^{-/-} fibroblasts. (H) Insulin-stimulated IRS-1 tyrosine phosphorylation and total IRS-1 levels following tunicamycin treatment (Tun, 10 μg/ml for 1 hour) in IRE-1α^{+/+} and IRE-1α^{-/-} fibroblasts. Quantitation of insulin-induced IRS-1 tyrosine phosphorylation levels in IRE-1α^{+/+} and IRE-1α^{-/-} cells is displayed in the bottom of the panel. All graphs show mean±SEM from at least 2 independent experiments and statistical significance from the controls is indicated by * with p<0.005.

FIG. 12 shows the inhibition of insulin receptor signaling by thapsigargin-induced ER stress and the role of Ca levels in IRS-1 serine phosphorylation. (A) ER stress was induced in Fao cells by 1 hour treatment with 300 nM thapsigargin (Thap), and cells were subsequently stimulated with insulin (Ins). IRS-1 tyrosine phosphorylation (pY) and serine phosphorylation (pSer307), insulin receptor (IR) tyrosine phosphorylation, and total protein levels were examined using either immunoprecipitation (IP) followed by immunoblotting (IB) or direct immunoblotting. (B) Fao cells were treated with sulindac sulfide (SS: 0, 7.5, 30, and 60 μM) for 45 minutes with or without an additional hour of exposure to 300 nM thapsigargin (Thap). IRS-1 serine phosphorylation and total IRS-1 protein levels were examined as described above.

FIG. 13 demonstrates the alteration of the ER stress response by manipulation of XBP-1 levels leads to alterations in insulin receptor signaling. ER stress responses in XBP-1s overexpressing cells, XBP-1^{-/-} cells and their controls. (A) Induction of XBP-1s expression upon removal of doxycycline in mouse embryonic fibroblasts (MEF). (B) Southern blot analysis of XBP-1^{-/-} MEF cells and their WT controls for the wild type (9.4 kb) and targeted (6.5 kb) alleles. (C) PERK phosphorylation (p-PERK) and JNK activity in the XBP-1s overexpressing cells and their control cells (−Dox and +Dox, respectively) upon tunicamycin treatment (Tun, 2 μg/ml). (D) PERK phosphorylation and JNK activity upon low dose tunicamycin treatment (Tun, 0.5 μg/ml) in XBP-1^{-/-} MEF cells and their WT controls. (E) IRS-1 Ser307 phosphorylation upon tunicamycin treatment (Tun, 2 μg/ml) in the XBP-1s overexpressing and the control cells (−Dox and +Dox, respectively), detected using immunoprecipitation (IP) of IRS-1 followed by immunoblotting (IB) with an IRS-1 phosphoserine 307-specific antibody. The graph next to the blots shows the quantitation of IRS-1 Ser307 phosphorylation under conditions described in panel E. (F) Insulin-stimulated tyrosine phosphorylation of IRS-1 in the XBP-1s overexpressing and controls cells with or without tunicamycin treatment (Tun, 2 μg/ml). The ratio of IRS-1 tyrosine phosphorylation to total IRS-1 level was summarized from independent experiments and was presented in the graph. (G) IRS-1 Ser307 phosphorylation upon tunicamycin treatment (Tun, 0.5 μg/ml) in XBP-1^{-/-} cells and WT controls was detected as described in panel C. The graph next to the blots shows the quantitation of IRS-1 Ser307 phosphorylation under conditions described in FIG. 12G. (H) Insulin-stimulated tyrosine phosphorylation of IRS-1 in XBP-1^{-/-} and WT control cells with or without tunicamycin treatment (Tun, 0.5 μg/ml). The ratio of IRS-1 tyrosine phosphorylation to total IRS-1 level was summarized from independent experiments and presented in the graph. All graphs show mean±SEM from at least 2 independent experiments and statistically significance from the controls is indicated by * with p<0.005.

FIG. 14 shows insulin-induced insulin receptor autophosphorylation in XBP-1 overexpressing and XBP-1-deficient cells. (A) XBP-1 overexpressing cells and their control MEF cells (−Dox and +Dox, respectively) were treated with 2 μg/ml tunicamycin (Tun) for various period (0, 0.5, 1, 2, 3, and 4 hours). Insulin-induced insulin receptor (IR) tyrosine phosphorylation (pY) and total IR levels were examined in those cells using immunoprecipitation (IP) with IR antibody followed by immunoblotting (IB) with antibodies against IR or phospho tyrosine (pY). (B) XBP-1^{-/-} MEF cells and their WT controls were treated with 0.5 μg/ml tunicamycin for various period (0, 0.5, 1, 2, 3, and 4 hours). Insulin-induced insulin receptor (IR) tyrosine phosphorylation (pY) and total IR levels were examined as in panel A.

FIG. 15 shows glucose homeostasis in XBP-1^{-/-} mice on high fat diet. The XBP-1^{+/-} (⋄) and XBP-1^{+/+} (▪) mice were placed on high fat diet (HFD) immediately after weaning. Total body weight (A), fasting blood insulin (B), C-peptide (C), and glucose (D) levels were measured in the XBP-1^{+/-} and XBP-1^{+/+} mice during the course of HFD. Glucose tolerance tests were performed after 7 (E) and 16 (F) weeks on HFD in XBP-1^{+/-} and XBP-1^{+/+} mice. Insulin tolerance tests were performed after 8 (G) and 17 (H) weeks on HFD in XBP-1^{+/-} and XBP-1^{+/+} mice. n=11 XBP-1^{+/-} mice; n=8 XBP-1^{+/+} mice. Data are shown as mean±SEM. Statistical significance in two-tailed student t test at p≦0.05 is indicated by *,p≦0.005 by ** and p≦0.0005 by ***. XBP-1^{+/-} and XBP-1^{+/+} groups are also compared by ANOVA (panels A-H).

FIG. 16 shows ER stress and insulin receptor signaling in XBP-1^{+/-} mice. PERK phosphorylation (p-PERK) (A), JNK activity (p-c-Jun) (B), and IRS-1 Ser307 (IRS-1^{pSer307}) (C) were examined in the livers of XBP-1^{+/-} and XBP-1^{+/+} mice after 16 weeks on high fat diet. After infusion of insulin (1 U/kg) through portal vein, insulin receptor (IR) tyrosine phosphorylation (pY) (D), IRS-1 tyrosine phosphorylation (E), IRS-2 tyrosine phosphorylation (F), and Akt Ser473 phosphorylation (G) were examined in livers of XBP-1^{+/-} and XBP-1^{+/+} mice after 16 weeks on high fat diet.

FIG. 17 is the characterization of pancreatic islets in XBP-1^{+/-} and XBP-1^{+/+} mice. Islet morphology, size, and immunohistochemical staining for insulin and glucagon in pancreatic sections obtained from XBP-1^{+/-} and XBP^{+/+} mice on either regular diet (A-D) or HFD (E-H). Glucose-stimulated insulin secretion in XBP-1^{+/-} and XBP-1^{+/+} mice on high fat diet Glucose-stimulated insulin secretion was examined in XBP-1^{+/-} and WT mice placed on high fat diet for 16 weeks (I). Glucose was administered introperitoneally to mice in each genotype and blood samples are collected at the indicated times for insulin measurements. In these experiments, there was no detectable abnormality in the XBP^{+/-} islets and no difference was evident between genotypes under standard conditions. On HFD, both the XBP-1^{+/-} and XBP-1^{+/+} mice exhibited islet hyperplasia. This anticipated response to HFD was similar between genotypes and the hyperplastic component (islet size >150 μM) comprised 40% of all islets in XBP-1^{+/-} and 43% of all islets in WT mice on HFD. In experiments examining glucose-stimulated insulin secretion in XBP-1^{+/-} and WT mice on HFD, the XBP-1^{+/-} mice responded to glucose with even a stronger insulin secretory response, which effectively eliminates the possibility of an isolated islet defect underlying their phenotype. Hence, these data indicate that the phenotype of the XBP-1^{+/-} mice cannot be explained by defective islets and even after 16 weeks on HFD, the islets appear indistinguishable between genotypes.

FIG. 18 shows ER stress and insulin receptor signaling in XBP-1^{+/-} mice. PERK phosphorylation (p-PERK) (A), JNK activity (p-c-Jun) (B), and IRS-1 Ser307 (IRS-1^{pSer307}) (C) were examined in the livers of XBP-1^{+/-} and XBP-1^{+/+} mice after 16 weeks on high fat diet. After infusion of insulin (1 U/kg) through portal vein, insulin receptor (IR) tyrosine phosphorylation (pY) (D), IRS-1 tyrosine phosphorylation (E), IRS-2 tyrosine phosphorylation (F), and Akt Ser473 phosphorylation (G) were examined in livers of XBP-1^{+/-} and XBP-1^{+/+} mice after 16 weeks on high fat diet.

FIG. 19 shows intact insulin receptor signaling in liver and adipose tissues of XBP-1^{+/-} and XBP-1^{+/+} mice on regular diet. After infusion of insulin (1 U/kg) through the portal vein, insulin receptor (IR) tyrosine phosphorylation (pY), IRS-1 tyrosine phosphorylation, IRS-2 tyrosine phosphorylation, Akt Ser473 phosphorylation, and their total protein levels were examined in livers (A) and adipose tissues (B) of XBP-1^{+/+} and XBP^{+/+} mice on regular diet.

FIG. 20 shows reduced insulin receptor signaling in adipose tissues of XBP-1^{+/-} and XBP-1^{+/+} mice on high fat diet. (A) After infusion of insulin (1 U/kg) through the portal vein, insulin receptor (IR) tyrosine phosphorylation (pY), IRS-1 tyrosine phosphorylation, IRS-2 tyrosine phosphorylation, Akt Ser473 phosphorylation, and their total protein levels were examined in adipose tissues of XBP-1^{+/-} and XBP^{+/+} mice on high fat diet for 16 weeks. (B) JNK kinase assay was performed in adipose tissues of XBP-1^{+/-} and XBP^{+/+} mice on high fat diet for 16 weeks.

FIG. 21 shows increased JNK activity in the liver tissue of obese mice followed by normalization of JNK activity after treatment with PBA.

### DETAILED DESCRIPTION OF CERTAIN PREFERRED EMBODIMENTS OF THE INVENTION

Endoplasmic reticulum (ER) stress has been found to be important in the pathogenesis of a variety of diseases including α1-anti-trypsin deficiency, urea cycle disorders, type 1 diabetes, and cystic fibrosis. The present invention stem from the recognition that ER stress is implicated in the pathogenesis of diseases such as obesity, peripheral insulin resistance, hyperglycemia, and type 2 diabetes (Ozcan et al., “Endoplasmic Reticulum Stress Link Obesity, Insulin Action, and Type 2 Diabetes” Science 306:457-461, 2004; incorporated herein by reference). Based on this discovery, agents that reduce or prevent ER stress have been shown to be useful in the treatment of obesity, insulin resistance, hyperglycemia, and type 2 diabetes.

Any agent known to reduce or modulate ER stress is useful in treating these metabolic diseases. These agents may act to reduce or prevent ER stress in any manner. In certain embodiments, the agent may increase the capacity of the ER to process proteins (e.g., increasing the expression of ER chaperones, increasing the levels of post-translational machinery). In other embodiments, the agent may reduce the quantity of proteins to be processed by the ER (e.g., decreasing the total level of protein produced in a cell, reducing the level of protein processed by the ER, reducing the level of mutant proteins, reducing the level of misfolded proteins). Yet other agents may cause the release of misfolded/mutant proteins from the ER. The agent may work in all cells, or the effect may be limited to certain cells type (e.g., secretory cells, epithelial cells, hepatocytes, adipocytes, endocrine cells, etc.). In certain embodiments, the agents are particularly useful in reducing ER stress in adipose cells. In other embodiments, the agents are particularly useful in reducing ER stress in hepatic cells. The agents may work on the transcriptional, translational, post-translational, or protein level to reduce or prevent ER stress.

The administration of an effective dose of an ER stress modulator, or a combination therapy including an ER stress modulator, to a subject to treat or prevent obesity, insulin resistance, type 2 diabetes, hyperglycemia, or other related disease may cure the disease being treated, alleviate or reduce at least one sign or symptoms of the disease being treated, reduce the short term consequences of the disease, reduce the long term consequences of the disease, or provide some other transient beneficial effect to the subject. In certain embodiments, the inventive treatment increases insulin sensitivity. In other embodiments, the inventive treatment decreases blood glucose levels. In other embodiments, the inventive treatment prevents the long term consequences of diabetes including atherosclerosis, diabetic retinopathy, peripheral neuropathy, etc. In certain embodiments, the inventive treatment reduces levels of ER stress markers (e.g., spliced froms of XBP-1, phosphorylation status of PERK, phosphorylation of eIF2α, mRNA levels of GRP78/BIP, protein levels of GRP78/BIP, JNK activity) in cells (e.g., adipocytes, hepatocytes). In certain embodiments, the inventive treatment increases insulin action. In other embodiments, the inventive treatment increases insulin receptor signalling (e.g., phosphorylation of insulin receptor, IRS-1, IRS-2, akt). In certain embodiments, the inventive treatment suppresses appetite. In other embodiments, the inventive treatment prevents weight gain or promotes weight loss. In certain embodiments, the inventive treatment prevents the development of type 2 diabetes. In certain embodiments, the inventive treatment prevents the development of obesity. In certain embodiments, the inventive treatment prevents the development of hyperglycemia.

The agent may be any type of chemical compound. The agent may be a small molecule, organometallic complex, an inorganic compound, a protein, a glycoprotein, a peptide, a carbohydrate, a lipid, or a nucleic acid. In certain embodiments, the agent is a small molecule. Particularly useful agents are known as chemical chaperones, which are known to stabilize proteins against denaturation thereby preserving the protein's structure and function. Chemical chaperones include glycerol, D₂O, dimethylsulfoxide (DMSO), 4-phenyl butyrate (PBA), tauroursodeoxycholic acid (TUDCA), glycine betaine (betaine), glycerolphosphocholine (GPC), methylamines, and trimethylamine N-oxide (TMAO). In certain embodiments, combinations of one or more chemical chaperones may be used. These chemical chaperones are administered in doses ranging from 10 mg/kg/day to 10 g/kg/day, preferably 100 mg/kg/day to 5 g/kg/day, more preferably from 500 mg/kg/day to 3 g/kg/day. In certain embodiments, the agent is administered in divided doses (e.g., twice per day, three times a day, four times a day, five times a day). In other embodiments, the agent is administered in a single dose per day.

The agent may be combined with one or more other pharmaceutical agents, particularly agents traditionally used in the treatment of diabetes, obesity, or insulin resistance. A list of agents useful in combination with ER stress modulators (e.g., PBA, TUDCA, TMAO, or derivatives thereof) is included as Appendix A. The list includes generic names, trade names, and manufacturers. Exemplary agents useful in combination with ER stress reducing agents include, but are not limited to, anti-diabetic agents (e.g., insulin, hypoglycemic agents (e.g., oral hypoglycemic agents such as sulfonylureas, tolbutamide, metformin, chlorpropamide, acetohexamide, tolazamide, glyburide, etc.)), anti-obesity agents, anti-dyslipidemia agent or anti-atherosclerosis agent (e.g., cholesterol lowering agents (e.g., HMg—CoA reductase inhibitors such as lovastatin, atorvastatin, simvastatin, pravastatin, fluvastatin, etc., aspirin), anti-obesity agent (e.g., appetite suppressants), vitamins, minerals, and anti-hypertensive agents.

In certain embodiments, a chemical chaperone or ER stress modulator (e.g., PBA, TUDCA, TMAO, or derivatives thereof) is used in combination with an anti-diabetic agent. Examplary anti-diabetic agents include biguanides (e.g., metformin), sulfonylureas (e.g., glimepiride, glyburide, glibenclamide, glipizide, gliclazide), insulin and analogs thereof (e.g., insulin lispro, insulin glargine, exubera, AERx insulin diabetes management system, AIR inhaled insulin, oralin, insulin detemir, insulin glulisine), peroxisome proliferator-activated receptor-gamma agonists (e.g., rosiglitazone, pioglitazone, isaglitazone, rivoglitazone, T-131, MBX-102, R-483 CLX-0921), dual PPAR agonists and PPAR pan agonists (e.g., BMS-398585, tesaglitazar, muraglitazar, naveglitazar, TAK-559, netoglitazone, GW-677594, AVE-0847, LY-929, ONO-5129), combination therapies (e.g., metformin/glyburide, metformin/rosiglitazone, metformin, glipizide), meglitinides (e.g., repaglinide, nateglinide), alpha-glucosidase inhibitors (e.g., acarbose, miglitol, voglibose), glucagon-like peptide-1 (GLP-1) analogues and agonists (e.g., Exenatide, Exenatide LAR, Liraglutide, CJC-1131, AVE-0010, BIM-51077, NN-2501, SUN-E7001), dipeptidyl peptidase IV (DPP-IV) inhibitors (e.g., LAF-237, MK-431 (Merck and Co), PSN-9301 (Probiodrug Prosidion), 815541 (GlaxoSmithKline-Tanabe), 823093 (GlaxoSmithKline), 825964 (GlaxoSmithKline), BMS-477118), pancreatic lipase inhibitors (e.g., orlistat), sodium glucose co-transporter (SGLT) inhibitors (e.g., T-1095 (Tanabe-J&J), AVE-2268, 869682 (GlaxoSmithKline-Kissei)), and amylin analog (e.g., pramlintide).

In other embodiments, a chemical chaperone or ER stress modulator (e.g., PBA, TUDCA, TMAO, or derivatives thereof) is used in combination with an anti-obesity agent. Exemplary anti-obesity agents include pancreatic lipase inhibitors (e.g., orlistat), serotonin and norepinephrine reuptake inhibitors (e.g., sibutramine), noradrenergic anorectic agents (e.g., phentermine, mazindol), peripherally acting agents (e.g., ATL-962 (Alizyme), HMR-1426 (Aventis), GI-181771 (GlaxoSmithKline)), centrally acting agents (e.g., Recombinant human ciliary neurotrophic factor, Rimonabant (SR-141716) (Sanofi-Synthélabo), BVT-933 (GlaxoSmithKline/Biovitrum), Bupropion SR (GlaxoSmithKline), P-57 (Phytopharm)), thermogenic agents (e.g., TAK-677 (AJ-9677) (Dainippon/Takeda)), cannabinoid CB1 antagonists (e.g., acomplia, SLV319), cholecystokinin (CCK) agonists (e.g., GI 181771 (GSK)), lipid metabolism modulator (e.g., AOD9604 (Monash University/Metabolic Pharmaceuticals), glucagon-like peptide 1 agonist (e.g., AC137 (Amylin)), leptin agonist (e.g., second generation leptin (Amgen), beta-3 adrenergic agonists (e.g., SR58611 (Sanofi-Aventis), CP 331684 (Pfizer), LY 377604 (Eli Lilly), n5984 (Nisshin Kyorin Pharmaceutical)), peptide hormone (e.g., peptide YY [3-36] (Nastech)), CNS modulator (e.g., S2367 (Shionogi & Co. Ltd.)), neurotrophic factor (e.g., peg axokine), and 5HT2C serotonin receptor agonist (e.g., APD356). Other anti-obesity agents include methamphetamine HCl, 1426 (Sanofi-Aventis), 1954 (Sanofi-Aventis), c-2624 (Merck & Co), c-5093 (Merck & Co), and T71 (Tularik).

In yet other embodiments, a chemical chaperone or ER stress modulator (e.g., PBA, TUDCA, TMAO, or derivatives thereof) is used in combination with an anti-dyslipidemia agent or anti-atherosclerosis agent. Exemplary anti-dyslipidemia agents or anti-atherosclerosis agents include HMG-CoA reductase inhibitors (e.g., atorvastatin, pravastatin, simvastatin, lovastatin, fluvastatin, cerivastatina, rosuvastatin, pitivastatin), fibrates (e.g., ciprofibrate, bezafibrate, clofibrate, fenofibrate, gemfibrozil), bile acid sequestrants (e.g., cholestyramine, colestipol, colesevelam), niacin (immediate and extended release), anti-platelets (e.g., aspirin, clopidogrel, ticlopidine), angiotensin-converting enzyme (ACE) inhibitors (e.g., ramipril, enalapril), angiotensin II receptor antagonists (e.g., losartan potassium), acyl-CoA cholesterol acetyltransferase (ACAT) inhibitors (e.g., avasimibe, eflucimibe, CS-505 (Sankyo and Kyoto), SMP-797 (Sumito)), cholesterol absorption inhibitors (e.g., ezetimibe, pamaqueside), nicotinic acid derivatives (e.g., nicotinic acid), cholesterol ester transfer protein (CETP) inhibitors (e.g., CP-529414 (Pfizer), JTT-705 (Japan Tobacco), CETi-1, torcetrapib), microsomal triglyceride transfer protein (MTTP) inhibitors (e.g., implitapide, R-103757, CP-346086 (Pfizer)), other cholesterol modulators (e.g., NO-1886 (Otsuka/TAP Pharmaceutical), CI-1027 (Pfizer), WAY-135433 (Wyeth-Ayerst)), bile acid modulators (e.g., GT102-279 (GelTex/Sankyo), HBS-107 (Hisamitsu/Banyu), BTG-511 (British Technology Group), BARI-1453 (Aventis), S-8921 (Shionogi), SD-5613 (Pfizer), AZD-7806 (AstraZeneca)), peroxisome proliferation activated receptor (PPAR) agonists (e.g., Tesaglitazar (AZ-242) (AstraZeneca), Netoglitazone (MCC-555) (Mitsubishi/Johnson & Johnson), GW-409544 (Ligand Pharmaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-518674 (Ligand Pharmaceuticals and Eli Lilly), MK-767 (Merck and Kyorin)), gene-based therapies (e.g., AdGVVEGF121.10 (GenVec), ApoA1 (UCB Pharma/Groupe Fournier), EG-004 (Trinam) (Ark Therapeutics), ATP-binding cassette transporter-A1 (ABCA1) (CV Therapeutics/Incyte, Aventis, Xenon)), composite vascular protectant (e.g. AGI-1067 (Atherogenics)), BO-653 (Chugai), glycoprotein IIb/IIIa inhibitors (e.g., Roxifiban (Bristol-Myers Squibb), Gantofiban (Yamanouchi), Cromafiban (Millennium Pharmaceuticals)), aspirin and analogs thereof (e.g., asacard, slow-release aspirin, pamicogrel), combination therapies (e.g., niacin/lovastatin, amlodipine/atorvastatin, simvastatin/ezetimibe), IBAT inhibitors (e.g., S-89-21 (Shionogi)), squalene synthase inhibitors (e.g., BMS-188494 I(Bristol-Myers Squibb), CP-210172 (Pfizer), CP-295697 (Pfizer), CP-294838 (Pfizer), TAK-475 (Takeda)), monocyte chemoattractant protein (MCP-1) inhibitors (e.g., RS-504393 (Roche Bioscience), other MCP-1 inhibitors (GlaxoSmithKline, Teijin, and Bristol-Myers Squibb)), liver X receptor agonists (e.g., GW-3965 (GlaxoSmithKline), TU-0901317 (Tularik)), and other new approaches (e.g., MBX-102 (Metabolex), NO-1886 (Otsuka), Gemeabene (Pfizer)).

In still other embodiments, a chemical chaperone or ER stress modulator (e.g., PBA, TUDCA, TMAO, or derivatives thereof) is used in combination with an anti-hypertensive agent. Examplary anti-hypertension agents include diurectics (e.g., chlorthalidone, metolazone, indapamide, bumetanide, ethacrynic acid, furosemide, torsemide, amiloride HCl, spironolactone, triamterene), alpha-blockers (e.g., doxazosin mesylate, prazosin HCl, terazosin HCl), beta-blockers (e.g., acebutolol, atenolol betaxolol, bisoprolol fumarate, carteolol HCl, metoprolol tartrate, metoprolol succinate, nadolol, penbutolol sulfate, pindolol, propanolol HCl, timolol maleate, carvedilol), Ca⁺² channel blockers (e.g., amlodipine besylate, felodipine, isradipine, nicardipine, nifedipine, nisoldipine, diltiazem HCl, verapamil HCl, azelnidipine, pranidipine, graded diltiazem formulation, (s)-amlodipine, clevidipine), angiotensin converting enzyme (ACE) inhibitors (e.g., benazepril hydrochloride, captopril, enalapril maleate, fosinopril sodium, lisinopril, moexipril, perindopril, quinapril hydrochloride, ramipril, trandolapril), angiotensin II (AT-II) antagonists (e.g., losartan, valsartan, irbesartan, candesartan, telmisartan, eprosartan, olmesarta, YM-358 (Yamanouchi)), vasopeptidase inhibitors (e.g. omapatrilat, gemopatrilat, fasidotril, sampatrilat, AVE 7688 (Aventis), M100240 (Aventis), Z13752A (Zambon/GSK), 796406 (Zambon/GSK)), dual neutral endopeptidase and enotheline converting enzyme (NEP/ECE) inhibitors (e.g., SLV306 (Solvay), NEP inhibitors (e.g., ecadotril), aldosterone antagonists (e.g., eplerenone), renin inhibitors (e.g., Aliskiren (Novartis), SPP 500 (Roche/Speedel), SPP600 (Speedel), SPP 800 (Locus/Speedel)), angiotensin vaccines (e.g., PMD-3117 (Protherics)), ACE/NEP inhibitors (e.g., AVE-7688 (Aventis), GW-660511 (Zambon SpA)), Na⁺/K⁺ATPase modulators (e.g., PST-2238 (Prassis-Sigma-Tau), endothelin antagonists (e.g., PD-156707 (Pfizer)), vasodilators (e.g., NCX-4016 (NicOx), LP-805 (Pola/Wyeth)), naturetic peptides (e.g., BDNP (Mayo Foundation)), angiotensin receptor blockers (ARBs) (e.g., pratosartan), ACE crosslink breakers (e.g., alagebrium chloride), endothelin receptor agonists (e.g., tezosentan (Genentech), ambrisentan (Myogen), BMS193884 (BMS), sitaxsentan (Encysive Pharmaceuticals), SPP301 (Roche/Speedel), Darusentan (Myogen/Abbott), J104132 (Banyu/Merck & Co.), TBC3711 (Encysive Pharmaceuticals), SB 234551 (GSK/Shionogi)), combination therapies (e.g., benazepril hydrochloride/hydrochlorothiazide, captopril/hydrochlorothiazide, enalapril maleate/hydrochlorothiazide, lisinopril/hydrochlorothiazide, losartan/hydrochlorothiazide, atenolol/chlorthalidone, bisoprolol fumarate/hydrochlorothiazide, metoprolol tartrate/hydrochlorothiazide, amlodipine besylate/benazepril hydrochloride, felodipine/enalapril maleate, verapamil hydrochloride/trandolapril, lercanidipine and enalapril, olmesartan/hydrochlorothiazide, eprosartan/hydrochlorothiazide, amlodipine besylate/atorvastatin, nitrendipine/enalapril), and MC4232 (University of Manitoba/Medicure).

In certain embodiments, a chemical chaperone or ER stress modulator (e.g., PBA, TUDCA, TMAO, or derivatives thereof) is used in combination with a vitamin, mineral, or other nutritional supplement.

In certain embodiments, the ER stress modulator (e.g., PBA, TUDCA, TMAO, or derivatives thereof) is administered in a sub-optimal dose (e.g., an amount that does not manifest detectable therapeutic benefits when administered in the absence of a second agent). In such cases, the administration of such an sub-optimal dose of the ER stress modulator in combination with another agent results in a synergistic effect. The ER stress modulator and other agent work together to produce a therapeutic benefit. In other embodiments, the other agent (i.e., not the ER stress modulator) is administered in sub-optimal doses. In combination with an ER stress modulator, the combination exhibits a therapeutic effect. In yet other embodiments, both the ER stress modulator and the other agent are administered in sub-therapeutic doses, and when combined produce a therapeutic effect. The dosages of the other agent may be below those standardly used in the art.

The dosages, route of administration, formulation, etc. for anti-diabetic agents, anti-obesity agents, anti-dyslipidemia agent or anti-atherosclerosis agent, anti-obesity agent, vitamins, minerals, and anti-hypertensive agents (listed above) are known in the art. The treating physician or health care professional may consult such references as the Physician's Desk Reference (59^{th} Ed., 2005), or Mosby's Drug Consult and Interactions (2005) for such information. It is understood that a treating physician would exercise his professional judgment to determine the dosage regimen for a particular patient.

The invention provides systems and methods of treating type 2 diabetes, insulin resistance, obesity, and other related conditions that provide a better therapeutic profile than the administration of the ER stress modality or the other treatment modality alone. In certain embodiments, the therapeutic effect may be greater. In certain embodiments, the combination has a synergistic effect. In other embodiments, the combination has an additive effect. The administration of a combination treatment regimen may reduce or even avoid certain unwanted or adverse side effects. In certain embodiments, the agents in the combination may be adminstered in lower doses, adminstered less frequently, or administered less frequently and in lower doses. Therefore, combination therapies with the above described benefits may increase patient compliance, improve therapy, and/or reduce unwanted or adverse side effects.

In certain embodiments, a chemical chaperone (e.g., PBA, TUDCA, TMAO, or derivatives thereof) is used in combination with a hypoglycemic agent. For example, insulin, glucagon, a biguanide hypoglycemic agent (e.g., metformin, phenformin, or buformin), a thiazolidinedione hypoglycemic agent (e.g., ciglitazone, pioglitazone), a sulfonylurea hypoglycemic agent (e.g., tolbutamide, chlorpropamide, acetohexamide, tolazamide, glyburide, glipizide, or gliclazide), an α-glucosidase inhibitor (e.g., acarbose), or diazoxide may be combined with glycerol, D₂O, dimethylsulfoxide (DMSO), 4-phenyl butyrate (PBA), tauroursodeoxycholic acid (TUDCA), glycine betaine (betaine), glycerolphosphocholine (GPC), methylamines, or trimethylamine N-oxide (TMAO). Certain specific exemplary combination therapies include insulin and PBA, insulin and TUDCA, insulin and betaine, insulin and GPC, insulin and TMAO, metformin and PBA, metformin and TUDCA, metformin and betaine, metformin and GPC, metformin and TMAO, a thiazolidinedione hypoglycemic agent and PBA, a thiazolidinedione hypoglycemic agent and TUDCA, a thiazolidinedione hypoglycemic agent and betaine, a thiazolidinedione hypoglycemic agent and GPC, and a thiazolidinedione hypoglycemic agent and TMAO. In certain embodiments, the combination used to treat or prevent obesity, insulin resistance hyperglycemia, or type 2 diabetes is 4-phenylbutyrate (PBA) and metformin. In terms of combination therapies whether they be combinations of chemical chaperones, combinations of ER stress modulators, or combinations of chemical chaperones/ER stress modulators and other agents such as hypoglycemic agents, the agents may be delivered concurrently or consecutively. In certain embodiments, the chemical chaperone or ER stress modulator is administered before the other agent. In other embodiments, the chemical chaperone or ER stress modulator is administered after the other agent.

In certain embodiments, small molecule agents shown to reduce ER stress include 4-phenyl butyrate (PBA), tauroursodeoxycholic acid (TUDCA), and trimethylamine N-oxide (TMAO). PBA is used currently to treat α1-anti-trypsin deficiency, urea cycle disorders, and cystic fibrosis. Derivatives, salts (e.g., sodium, magnesium, potassium, magnesium, ammonium, etc.), prodrugs, esters, isomers, and stereoisomers of PBA, TUDCA, or TMAO may also be used to treat obesity, hypergylcemia, type 2 diabetes, and insulin resistance. Without wishing to be bound by any particular theory, these compounds are thought to work by allowing the ER to better handle misfolded and/or mutant proteins being processed by the ER.

In certain embodiments, a derivative of 4-phenyl butyrate useful in the present invention is of the formula:

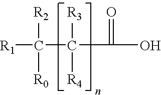

wherein n is 1 or 2;

R₀ is aryl, heteroaryl, or phenoxy, wherein the aryl, heteroaryl, and phenoxy being unsubstituted or substituted with, independently, one or more halogen, hydroxy, or lower alkyl (C₁-C₆) groups;

R₁ and R₂ are independently H, lower alkoxy, hydroxy, lower alkyl or halogen; and

R₃ and R₄ are independently H, lower alkyl, lower alkoxy or halogen; or

a pharmaceutically-acceptable salt thereof; or a mixture thereof. In certain embodiments, R₀ is a substituted or unsubstituted phenyl ring. In certain embodiments, R₀ is an unsubstituted phenyl ring. In other embodiments, R₀ is a monosubstituted phenyl ring. In yet other embodiments, R₀ is a disubstituted phenyl ring. In still other embodiments, R₀ is a trisubstituted phenyl ring. In certain embodiments, R₀ is a phenyl ring substituted with 1, 2, 3, or 4 halogen atoms. In certain embodiments, R₀ is a substituted or unsubstituted heteroaryl ring. In certain embodiments, R₀ is a naphthyl ring. In certain embodiments, R₀ is five- or six-membered, preferably six-membered. In certain embodiments, R₁ and R₂ are both hydrogen. In certain embodiments, n is 1. In other embodiments, n is 2. In certain embodiments, all R₃ and R₄ are hydrogen. In other embodiments, at least one R₃ or R₄ is hydrogen. In certain embodiments, the compound is used in a salt form (e.g., sodium salt, potassium salt, magnesium salt, ammonium salt, etc.) Other derivatives useful in the present invention are described in U.S. Pat. No. 5,710,178, which is incorporated herein by reference. 4-phenyl butyrate or its derivatives may be obtained from commercial sources, or prepared by total synthesis or semi-synthesis.

In certain embodiments, a derivative of TUDCA useful in the present invention is of the formula:

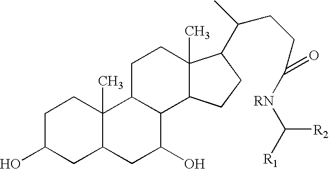

wherein:
R is —H or C₁-C₄ alkyl;
R₁ is —CH₂—SO₃R₃ and R₂ is —H; or R₁ is —COOH and R₂ is —CH₂—CH₂—CONH₂, —CH₂—CONH₂, —CH₂—CH₂—SCH₃ or —C₂—S—CH₂—COOH; and
R₃ is —H or a basic amino acid; or a pharmaceutically acceptable salt thereof. In certain embodiments, the stereochemistry of the derivative is defined as shown in the following structure:

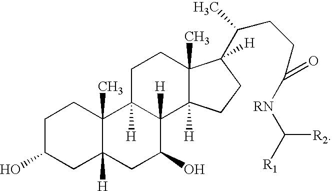

In certain embodiments, R is H. In other embodiments, R is methyl, ethyl, n-propyl, iso-propyl, n-butyl, iso-butyl, or tort-butyl, preferably, methyl. In certain embodiments, R₁ or R₂ is hydrogen. In certain embodiments, R₁ is —CH₂—SO₃R₃ and R₂ is —H. In other embodiments, R₁ is —COOH and R₂ is —CH₂—CH₂—CONH₂, —CH₂—CONH₂, —CH₂—CH₂—SCH₃ or —CH₂—S—CH₂—COOH. In certain embodiments, R₃ is hydrogen. In certain embodiments, R3 is lysine, arginine, ornithine, or histidine. Derivatives of TUDCA and ursodeoxycholic acid may be obtained from commercial sources, prepared from total synthesis, or obtained from a semi-synthesis. In certain embodiments, the derivative is prepared via semi-synthesis, for example, as described in U.S. Pat. Nos. 5,550,421 and 4,865,765, each of which is incorporated herein by reference.

In certain embodiments, derivative of trimethylamine N-oxide useful in the present invention is of the formula:

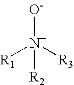

wherein

R₁, R₂, and R₃ are independently hydrogen, halogen, or lower C₁-C₆ alkyl; or

a pharmaceutically-acceptable salt thereof; or a mixture thereof. In certain embodiments, R₁, R₂, and R₃ are the same. In other embodiments, at least one of R₁, R₂, and R₃ is different. In yet other embodiments, all of R₁, R₂, and R₃ are different. In certain embodiments, R₁, R₂, and R₃ are independently hydrogen or lower C₁-C₆ alkyl. In yet other embodiments, R₁, R₂, and R₃ are independently lower C₁-C₆ alkyl. In still other embodiments, R₁, R₂, and R₃ are independently methyl, ethyl, or propyl. In certain embodiments, R₁, R₂, and R₃ are ethyl. Derivatives of TMAO may be obtained from commercial sources, or prepared by total synthesis or semi-synthesis.

In other embodiments, the agent is a nucleic acid, e.g., an inhibitory RNA such as an siRNA. In other embodiments, the agent is a protein, e.g., an antibody or antibody fragment. In yet other embodiments, the agent is a peptide.

In treating an animal, suffering from obesity, peripheral insulin resistance, hyperglycemia, or type 2 diabetes, a therapeutically effective amount of the agent is administered to the subject via any route to achieve the desired biological result. Any route of administration may be used including orally, parenterally, intravenously, intraarterially, intramuscularly, subcutaneously, rectally, vaginally, transdermally, intraperitoneally, and intrathecally. In certain embodiments, the agent is administered parenterally. In other embodiments, the agent is administered orally.

In the use of PBA, TUDCA, or TMAO, the agent is preferably administered orally; however, any of the administration routes listed above may also be used. In certain embodiments, the PBA, TUDCA, or TMAO is administered parenterally. PBA is administered in doses ranging from 10 mg/kg/day to 5 g/kg/day, preferably from 100 mg/kg/day to 1 g/kg/day, more preferably from 250 mg/kg/day to 750 mg/kg/day. TUDCA is administered in doses ranging from 10 mg/kg/day to 5 g/kg/day, preferably from 100 mg/kg/day to 1 g/kg/day, more preferably from 250 mg/kg/day to 750 mg/kg/day. TMAO is administered in doses ranging from 10 g/kg/day to 0.1 g/kg/day, preferably from 5 g/kg/day to 0.5 g/kg/day, more preferably from 2.5 g/kg/day to 500 mg/kg/day. In certain embodiments, the agent is administered in divided doses (e.g., twice per day, three times a day, four times a day, five times a day). In other embodiments, the agent is administered in a single dose per day.

### Pharmaceutical Compositions

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material, or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), transdermally, subcutaneously, bucally, or as an oral or nasal spray.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

The pharmaceutical compositions of the invention may be provided in a kit with other agents used to treat diabetes, insulin resistance, or obesity. The kit may include instructions for the treating physician and/or patient, which may include dosing information, safety information, list of side effects, chemical formula of agent, mechanism of action, etc. In certain embodiments, the kit may include materials for administering the pharmaceutical composition. For example, the kit may include a syringe, needle, alcohol swaps, etc. for the administration of an injectable preparation. In certain embodiments when two or more agents are provided in a kit, the active pharmaceutical ingredients may be formulated separately or together. For example, the kit may include a first container with as ER stress modulator (e.g., PBA, TUDCA, TMAO, or a derivative thereof) and a second container with a second agent used in treating type 2 diabetes, insulin resistance, hyperglycemia, obesity, or a related disorder (e.g., anti-diabetic agents, anti-obesity agents, anti-dyslipidemia agent or anti-atherosclerosis agent, anti-obesity agent, vitamins, minerals, and anti-hypertensive agents, as described above). In certain embodiments, the active pharmaceutical ingredients are formulated separately. In other embodiments, the active pharmaceutical ingredients are formulated together.

### Screening for ER Stress Reducers

As demonstrated herein, ER stress has been identified as a target for the treatment of various diseases including obesity, type 2 diabetes, insulin resistance, and hyperglycemia. Markers of ER stress have also been identified. With the need for new pharmaceutical agents that reduce or prevent ER stress, a method of identifying or screening for ER stress modulators is needed.

In certain embodiments, a chemical compound or a collection of chemical compounds is assayed to identify compounds that reduce or modulate ER stress in vivo or in vitro, preferably in vivo. These compounds may be any type of chemical compound including small molecules, proteins, peptides, polynucleotides, carbohydrates, lipids, etc. In certain embodiments, a collection of compounds is screened using the inventive method. These collections may be historical libraries of compounds from pharmaceutical companies. The collection may also be a combinatorial library of chemical compounds. The collection may include at least 5, 10, 50, 100, 500, 1000, 10000, 100000, or 1000000 compounds.

The compounds are contacted with cells. The cells may be any type of cells with an endoplasmic reticulum. The cells may be animal cells, plant cells, or fungal cells. In certain embodiments, mammalian cells are preferred, particularly human cells. The cells may be derived from any organ system. In certain embodiments, cells from adipose tissue or liver tissue are preferred.

In screening for agents that reduce ER stress, the test compound is contacted with a cell already experiencing ER stress. The ER stress in the cell may be caused by any techniques known in the art. For example, ER stress may be due to a genetic alteration in the cells (e.g., XBP-1 mutations) or the treatment with a chemical compound known to cause ER stress (e.g., tunicamycin, thapsigargin). The level of ER stress markers is assayed before and after addition of the test compound to determine if the compound reduces ER stress. Markers of ER stress that may be assayed in the inventive method include spliced forms of XBP-1, the phosphorylation status of PERK (e.g., Thr980), the phosphorylation status of eIF2α (e.g., Ser51), mRNA and/or protein levels of GRP78/BIP, and JNK activity. In certain embodiments, one ER marker is measured. In other embodiments, the levels of a combination of two, three, four, five, six, or more ER stress markers are measured. Test compounds that reduce the levels of ER stress markers by at least about 10%, 20%, 30%, 40%, 50%, 60%, 70%, 80%, 90%, 95%, 99%, or 100%, preferably at least 25%, more preferably at least 50%, are considered useful for evaluation of ER stress reducers in the clinic. As would be appreciated by one of skill in this art, the test compound may be tested at various concentrations and under various conditions (e.g., various cell types, various causes of ER stress (genetic vs. chemical), various formulations).

In another aspect, the invention provides for a method of identifying compounds that prevent ER stress. In screening for compounds that prevent ER stress, the cells are not experiencing ER stress before they are contacted with the test compound. After the cells are contacted with the test compound, an agent known to cause ER stress is added to the cells, and then the level of at least one ER markers is measured to determine whether the compound is able to prevent ER stress. As would be appreciated by one of skill in this art, the test compound may be tested at various concentrations and under various conditions.

Agents identified by the methods of the invention may be further tested for toxicity, pharmacokinetic properties, use in vivo, etc. so that they may be formulated and used in the clinic to treat obesity, type 2 diabetes, hyperglycemia, and insulin resistance. The identified agents may also find use in the treatment of other diseases associated with ER stress.

Diagnosing Conditions Associated with ER Stress

The identification of various ER stress markers allows for the diagnosis of conditions associated with ER stress and the screening of subjects at risk for developing conditions associated with ER stress. Obesity, hyperglycemia, type 2 diabetes, and insulin resistance have all been shown to be associated with ER stress. Therefore, measuring the level of an ER stress marker(s) in a subject allows for determining whether a patient is at risk for any of these conditions. Measuring the levels of ER stress markers may be used to determine the risk of developing any conditions associated with ER stress (e.g., cystic fibrosis, Alzheimer's Disease).

ER stress markers that have been identified include spliced forms of XBP-1, the phosphorylation status of PERK, the phosphorylation status of eIF2α, mRNA levels of GRP78/BIP, protein levels of GRP78/BIP, and JNK activity. These ER stress markers may be measured using any techniques known in the art for measuring mRNA levels, protein levels, protein activity, or phosphorylation status. Exemplary techniques for measuring ER stress markers include western blot analysis, northern blot analysis, immunoassays, quantitative PCR analysis, and enzyme activity assay (for a more detailed description of these techniques, please see Ausubel et al. Current Protocols in Molecular Biology (John Wiley & Sons, Inc., New York, 1999); Molecular Cloning: A Laboratory Manual, 2nd Ed., ed. by Sambrook, Fritsch, and Maniatis (Cold Spring Harbor Laboratory Press: 1989); each of which is incorporated herein by reference).

In determining whether a subject is at risk for a condition associated with ER stress, one may determine the levels of one, two, three, four, five, or six ER stress markers. In certain embodiments, the level of only one ER stress marker is determined. In other embodiments, the levels of at least two ER stress markers are determined. In yet other embodiments, the levels of at least three ER stress markers are determined.

Typically, if the level of an ER stress marker is determined to be increased as compared to a normal control, the subject tested is considered at risk for insulin resistance, obesity, hyperglycemia, or type 2 diabetes. The identified subject may then be subject to further testing, treatment may be begun, or the subject may be watched for the future development of symptoms and signs associated with insulin resistance, obesity, hyperglycemia, or type 2 diabetes. The next course of action is typically determined by the subject's health care provider in consultation with the subject.

The levels of ER stress markers may be determined for any cells in the subject's body. Preferably, the cells are connected to the condition being test for. For example, in testing for a condition such as obesity, type 2 diabetes, insulin resistance, or hyperglycemia, hepatocytes or adipocytes may be used. In certain embodiments, hepatocytes are used. In other embodiments, the cells are adipocytes. The cells may be obtained from the subject by liver biopsy in the case of hepatocytes. Adipocytes may be obtained by biopsy of the subject.

The invention also provides kits and systems for measuring the levels of various ER stress markers in a subject. The kit may include primers, hybridization probes, polynucleotides, antibodies, antibody fragments, gels, buffers, enzyme substrates, ATP or other nucleotides, tools for obtaining cells or a biopsy from the subject, instructions, software, etc. These materials for performing the diagnostic method may be conveniently packaged for use by a physician, scientist, pathologist, nurse, lab technician, or health care professional.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Endoplasmic Reticulum Stress Links Obesity, Insulin Action, and Type 2 Diabetes

### Results

### Induction of ER Stress in Obesity

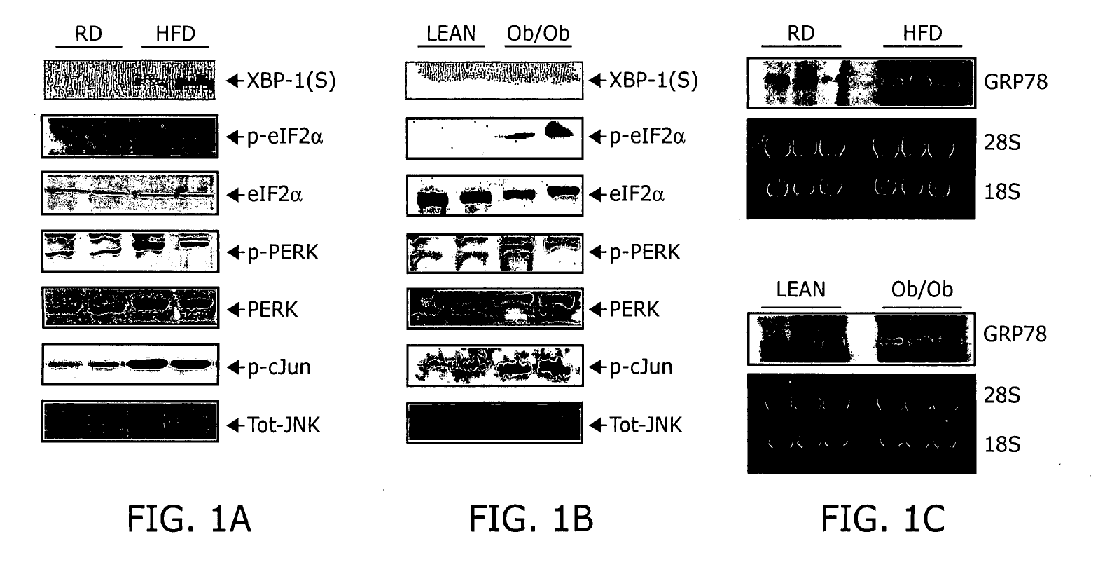

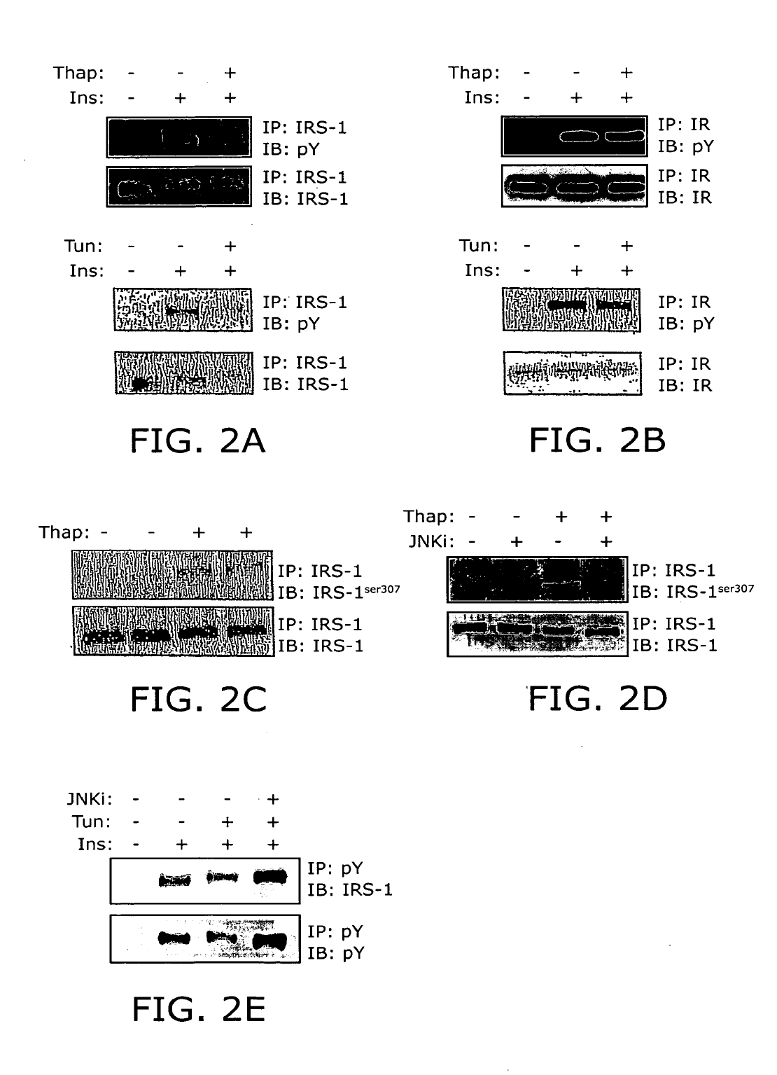

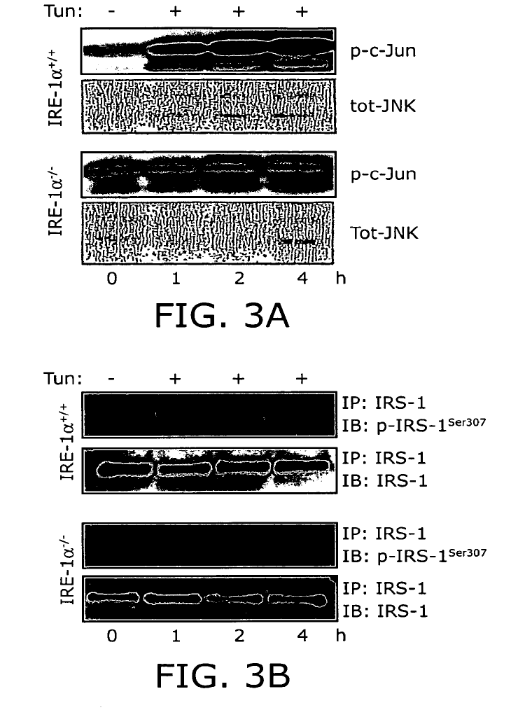

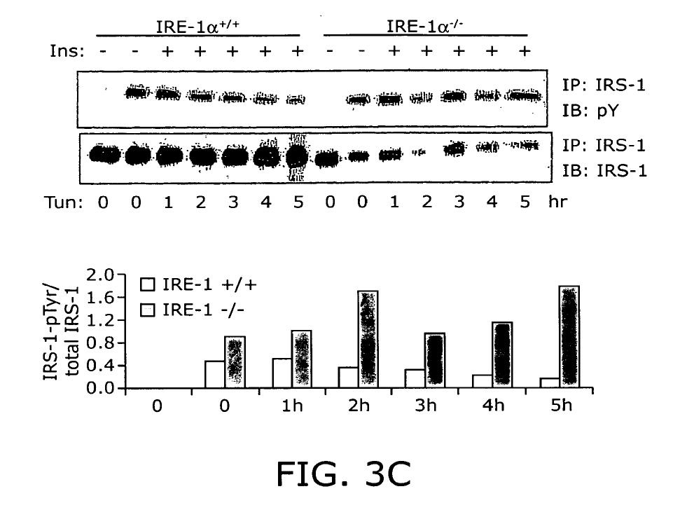

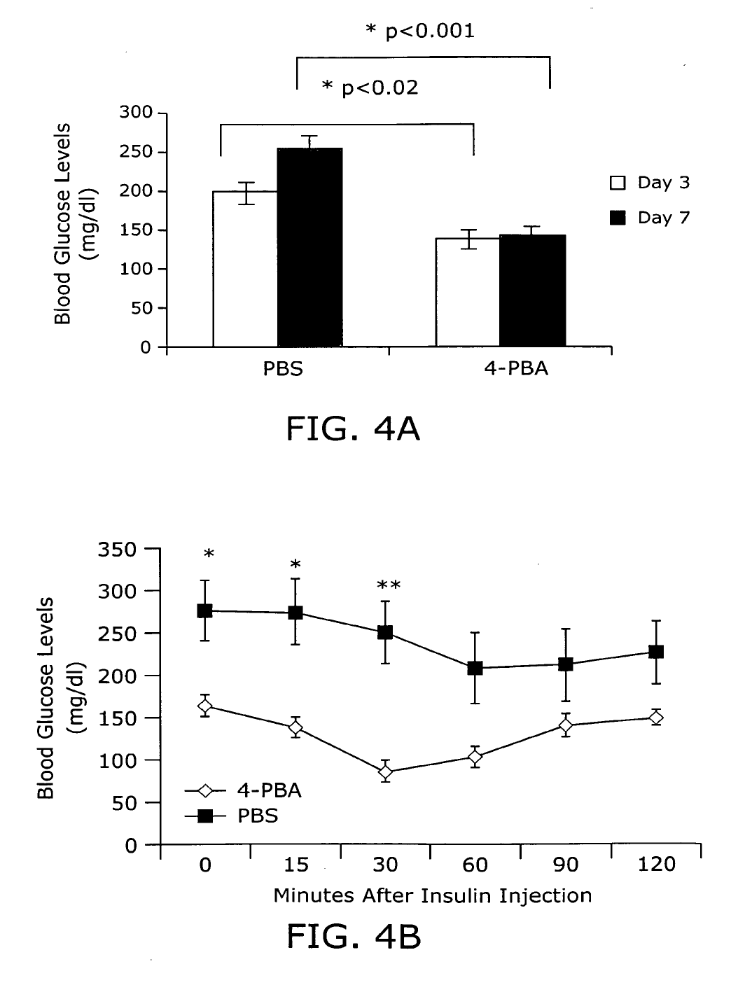

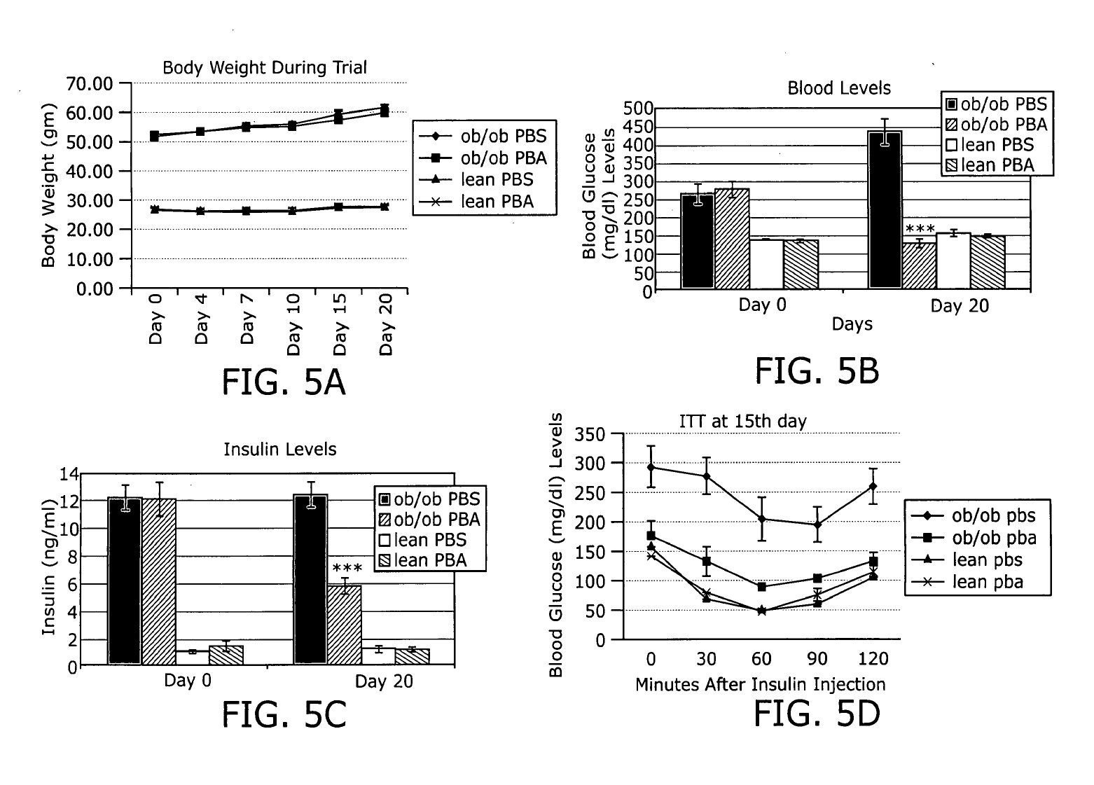

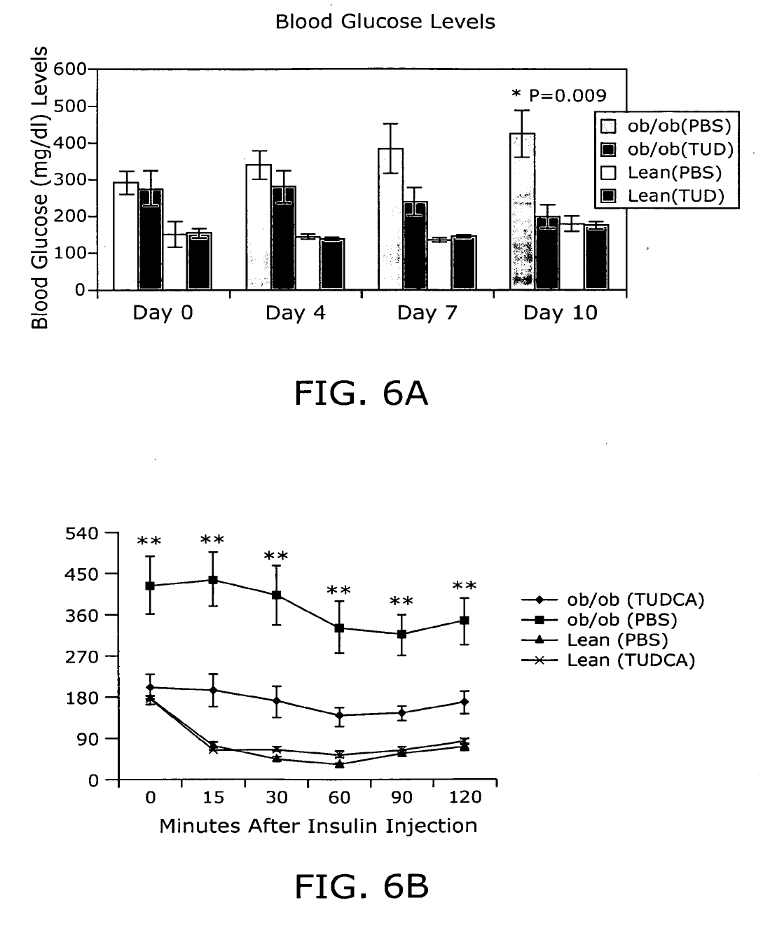

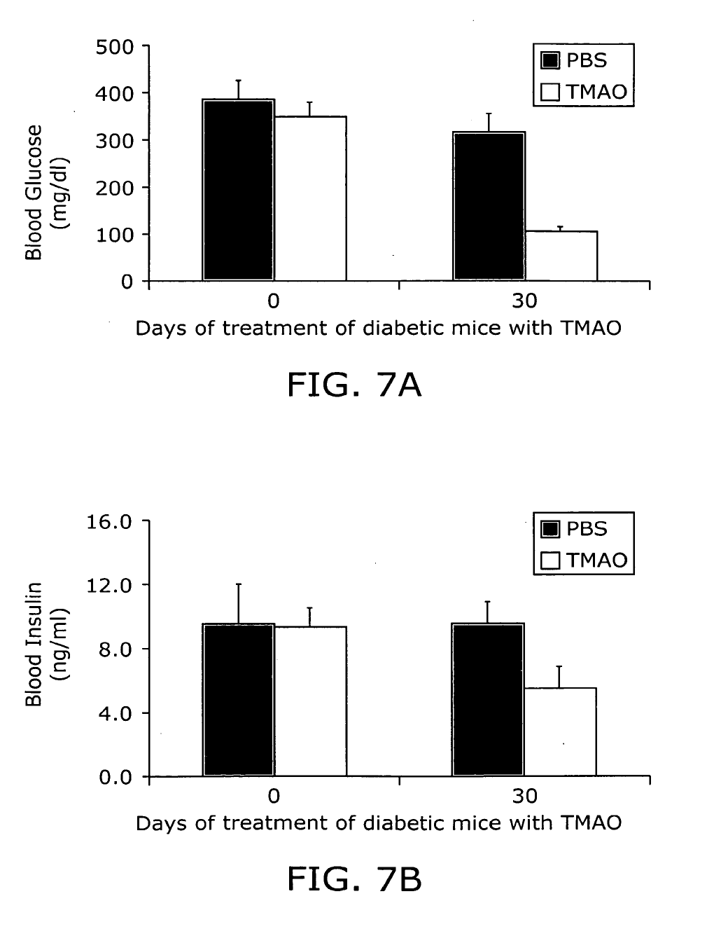

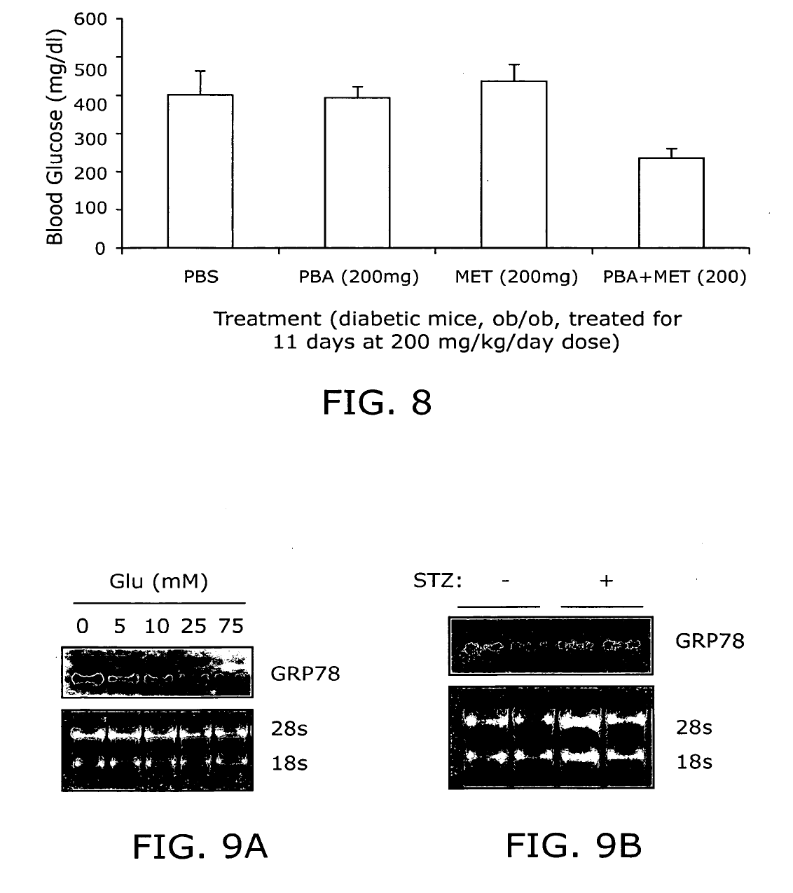

To examine whether ER stress is increased in obesity, we investigated the expression patterns of several molecular indicators of ER stress in dietary (high fat diet-induced) and genetic (ob/ob) models of murine obesity. The pancreatic ER kinase or PKR like kinase (PERK) is an ER transmembrane protein kinase that phosphorylates the α subunit of translation initiation factor 2 (eIF2α) in response to ER stress (Shi et al., Mol. Cell. Biol. 18, 7499 (1998); Harding et al., Nature 397, 271 (Jan. 21, 1999); each of which is incorporated herein by reference). The phosphorylation status of PERK and eIF2α is therefore a key indicator of the presence of ER stress. We determined the phosphorylation status of PERK (Thr980) and eIF2α (Ser51) using phospho-specific antibodies. These experiments demonstrated increased PERK and eIF2α phosphorylation in liver extracts of obese mice compared with lean controls (FIGS. 1A and 1B). ER stress also leads to JNK activation. Consistent with earlier observations (J. Hirosumi et al., Nature 420, 333 (2002); incorporated herein by reference), total JNK activity, indicated by c-Jun phosphorylation, was also dramatically elevated in the obese mice (FIGS. 1A and 1B).

The 78 kDa glucose regulated/binding Ig protein (GRP78/BIP) is an ER chaperone whose expression is increased upon ER stress. The GRP78/BIP mRNA levels were elevated in the liver tissue of obese mice compared with matched lean controls (FIGS. 1C and 1D). Since GRP78 expression is responsive to glucose, we tested whether this upregulation might simply be due to increasing glucose levels. Treatment of cultured rat Fao liver cells with high levels of glucose resulted in reduced GRP78 expression (FIG. 9A). Similarly GRP78 levels were not increased in a mouse model of hyperglycemia (FIG. 9B), indicating that regulation in obesity is unlikely to be related to glycemia alone.

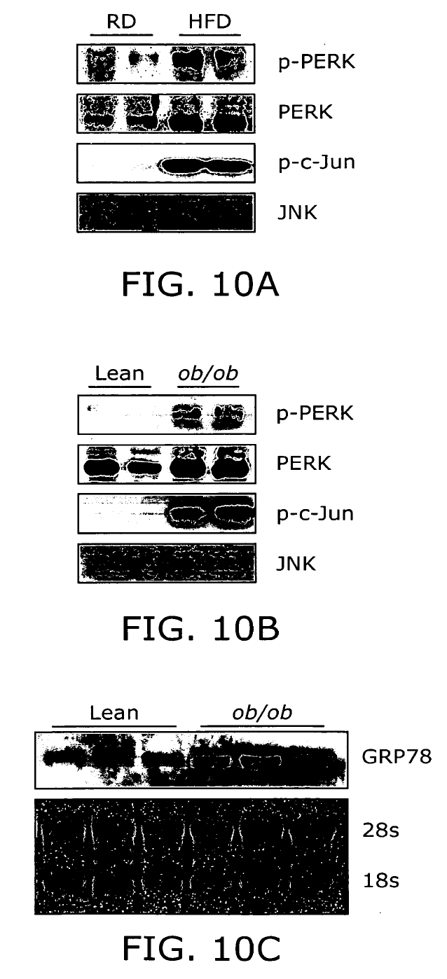

We also tested adipose and muscle tissues, important sites for metabolic homeostasis, for indications of ER stress in obesity. Similar to liver, PERK phosphorylation, JNK activity, and GRP78 expression were all significantly increased in adipose tissue of obese animals compared with lean controls (FIG. 10A-C). However, no indication for ER stress was evident in the muscle tissue of obese animals (data not shown). Taken together, these results indicate that obesity is associated with induction of ER stress predominantly in liver and adipose tissues.

### ER Stress Inhibits Insulin Action in Liver Cells

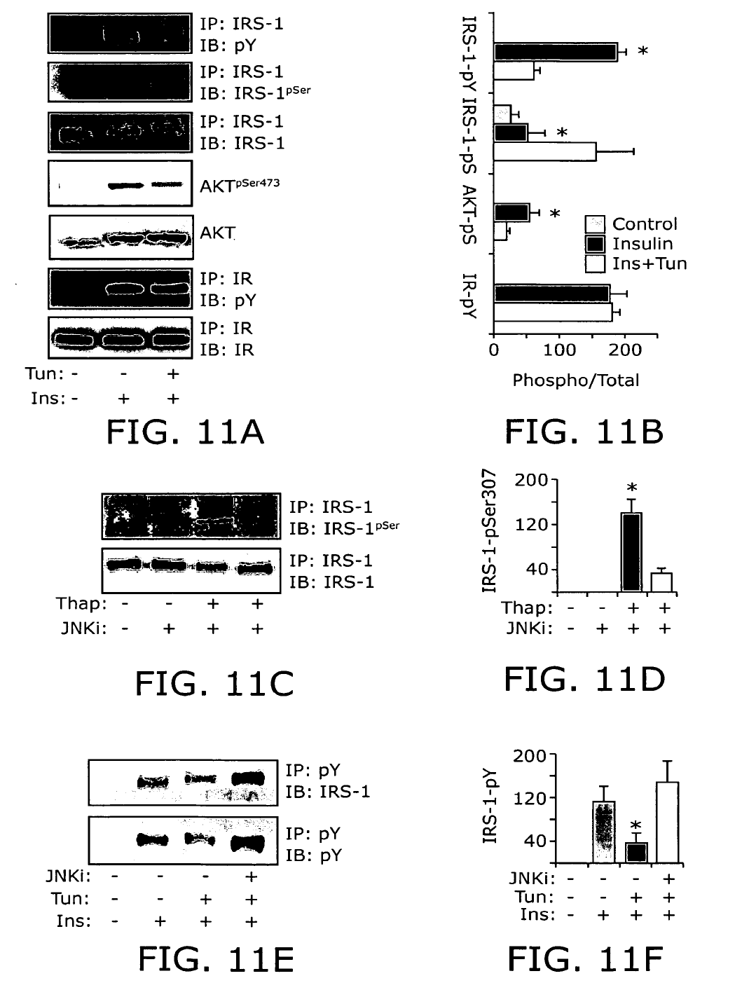

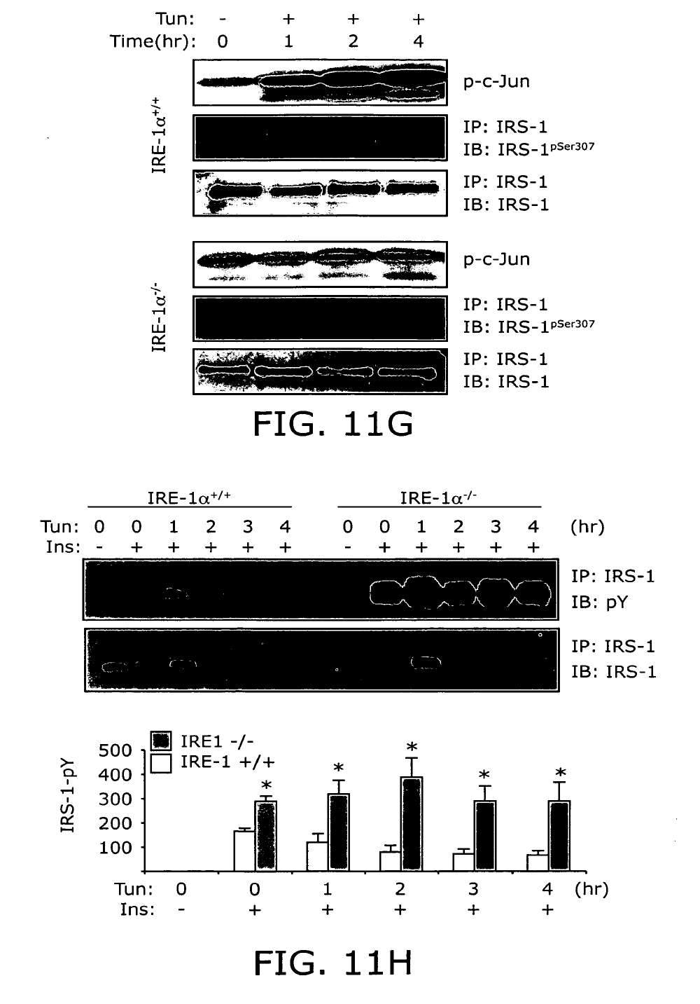

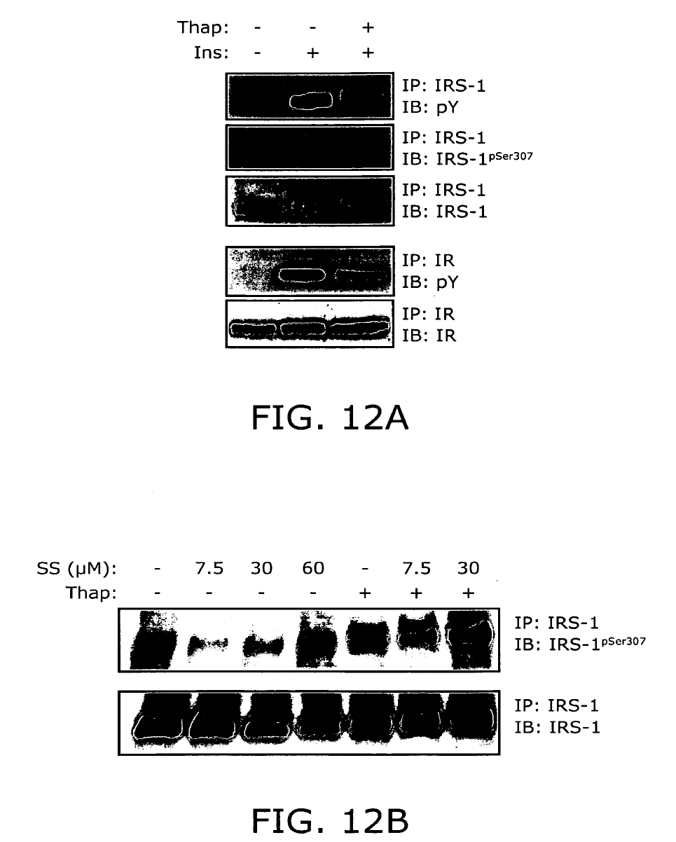

To investigate whether ER stress interferes with insulin action, we pretreated Fao liver cells with tunicamycin and thapsigargin, agents commonly used to induce ER stress. Tunicamycin significantly decreased insulin-stimulated tyrosine phosphorylation of IRS-1 (FIGS. 11A and 11B) and it also produced an increase in the molecular weight of IRS-1 (FIG. 11A). IRS-1 is a substrate for insulin receptor tyrosine kinase and serine phosphorylation of IRS-1, particularly mediated by JNK, reduces insulin receptor signaling (Hirosumi et al., Nature 420:333 (2002); incorporated herein by reference). Pretreatment of Fao cells with tunicamycin produced a significant increase in serine phosphorylation of IRS-1 (FIGS. 11A and 11B). Tunicamycin pretreatment also suppressed insulin-induced Akt phosphorylation, a more distal event in insulin receptor signaling pathway (FIGS. 11A and 11B). Similar, results were also obtained following treatment with thapsigargin (FIG. 12A), which was independent of alterations in cellular calcium levels (FIG. 12B).

We next examined the role of JNK in IRS-1 serine phosphorylation and inhibition of insulin-stimulated IRS-1 tyrosine phosphorylation by ER stress (FIGS. 11C and 11D). Inhibition of JNK activity with the synthetic inhibitor, SP600125, reversed the ER stress-induced serine phosphorylation of IRS-1 (FIGS. 11C and 11D). Pretreatment of Fao cells with a highly specific inhibitory peptide derived from the JNK binding protein, JIP (Barr et al., J. Biol. Chem. 277:10987 (2002); incorporated herein by reference), also completely preserved insulin receptor signaling in cells exposed to tunicamycin (FIGS. 11E and 11F). Similar results were obtained with the synthetic JNK inhibitor, SP600125 (data not shown). These results indicate that ER stress promotes a JNK-dependent serine phosphorylation of IRS-1, which in turn inhibits insulin receptor signaling.

### IRE-1 Plays a Crucial Role in Insulin Receptor Signaling

In the presence of ER stress, increased phosphorylation of inositol requiring kinase-1α (IRE-1α) leads to recruitment of TNF-α receptor-associated factor 2 (TRAF2) protein and activates JNK (Urano et al., Science 287:664 (2000); incorporated herein by reference). To address whether ER stress-induced insulin resistance is dependent on intact IRE-1α, we measured JNK activation, IRS-1 serine phosphorylation, and insulin receptor signaling upon exposure of IRE-1α^{-/-} and wild type (WT) fibroblasts to tunicamycin. In the WT but not IRE-1α^{-/-} cells, induction of ER stress by tunicamycin resulted in strong activation of JNK (FIG. 11G). Tunicamycin also stimulated phosphorylation of IRS-1 at Ser307 residue in WT (FIG. 11G) but not IRE-1α^{-/-} fibroblasts (FIG. 11E). Importantly, tunicamycin inhibited insulin-stimulated tyrosine phosphorylation of IRS-1 in the WT cells, whereas no such effect was detected in the IRE-1α^{-/-} cells (FIG. 11H). The level of insulin-induced tyrosine phosphorylation of IRS-1 was dramatically higher in IRE-1α^{-/-} cells despite lower total IRS-1 protein levels (FIG. 11H). These results demonstrate that ER stress-induced inhibition of insulin action is mediated by an IRE-1α-JNK-dependent protein kinase cascade.

### Manipulation of XBP-1 Levels Alters Insulin Receptor Signaling

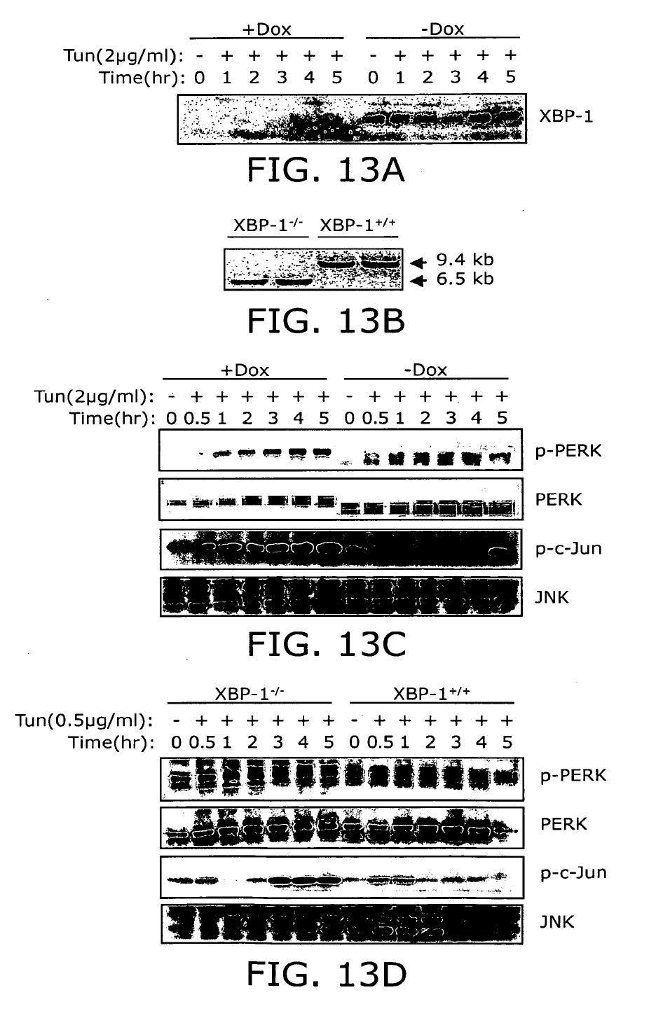

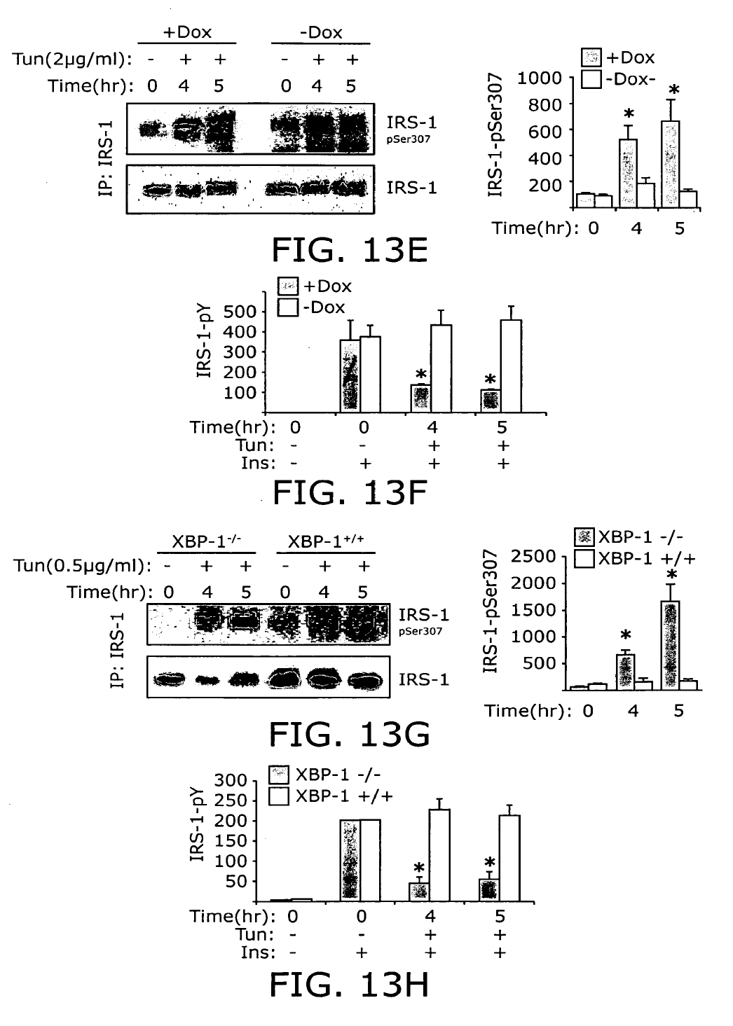

The transcription factor XBP-1 is a bZIP protein. The spliced or processed form of XBP-1 (XBP-1s) is a key factor in the transcriptional regulation of molecular chaperones and enhances the compensatory UPR (Calfon et al., Nature 415, 92 (2002); Shen et al., Cell 107:893 (2001); Yoshida et al., Cell 107:881 (2001); Lee et al., Mol. Cell Biol. 23:7448 (2003); each of which is incorporated herein by reference). We therefore reasoned that modulation of XBP-1s levels in cells should alter insulin action via its potential impact on the magnitude of the ER stress responses. To test this possibility, we established XBP-1 gain- and loss-of-function cellular models. First, we established an inducible gene expression system where exogenous XBP-1s is expressed only in the absence of tetracycline/doxycycline (FIG. 13A). In parallel, we also studied MEFs derived from XBP-1^{-/-} mouse embryos (Lee et al., Mol. Cell Biol. 23, 7448 (2003); A. M. Reimold et al., Genes Dev. 14, 152 (2000); Reimold et al., Nature 412, 300 (2001); each of which is incorporated herein by reference) (FIG. 13B). In fibroblasts without exogenous XBP-1s expression, tunicamycin treatment (2 μg/ml) resulted in strong PERK phosphorylation starting at 30 minutes and peaking at 3-4 hours associated with a mobility shift characteristic of PERK phosphorylation (FIG. 13C). In these cells, there was also a rapid and robust activation of JNK in response to ER stress (FIG. 13C). Upon induction of XBP-1s expression, there was a dramatic reduction in both PERK phosphorylation and JNK activation following tunicamycin treatment (FIG. 13C). Hence, overexpression of XBP-1s rendered WT cells refractory to ER stress. Similar experiments performed in XBP-1^{-/-} MEFs revealed an opposite pattern (FIG. 13D). XBP-1^{-/-} MEFs mounted a strong ER stress response even when treated with a low dose of tunicamycin (0.5 μg/ml), that failed to stimulate significant ER stress in WT cells (FIG. 13D). Under these conditions, PERK phosphorylation and JNK activation levels in XBP-1^{-/-} MEFs were significantly higher than those seen in WT controls (FIG. 13D), indicating that XBP-1^{-/-} cells are prone to ER stress. Thus alterations in the levels of cellular XBP-1s protein result in alterations in the ER stress response.

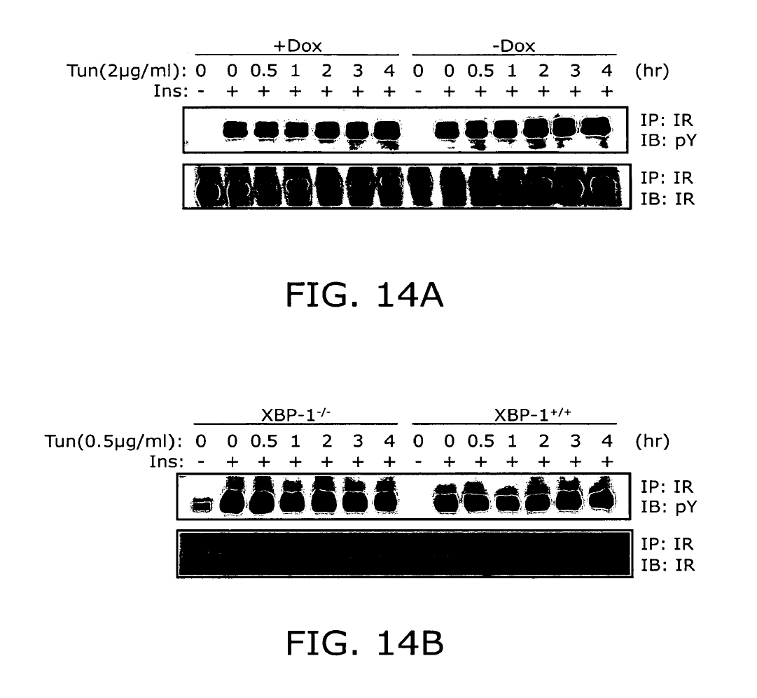

Next, we examined whether these differences in the ER stress responses produced alterations in insulin action as assessed by IRS-1 serine phosphorylation and insulin-stimulated IRS-1 tyrosine phosphorylation. Tunicamycin-induced IRS-1 serine phosphorylation was significantly reduced in fibroblasts exogenously expressing XBP-1s, compared with that of control cells (FIG. 13E). Upon insulin stimulation, the extent of IRS-1 tyrosine phosphorylation was significantly higher in cells overexpressing XBP-1s, compared with controls (FIG. 13F). In contrast, IRS-1 serine phosphorylation was strongly induced in XBP-1^{-/-} MEFs compared with XBP-1^{+/+} controls even at low doses of tunicamycin treatment (0.5 μg/ml) (FIG. 13G). Following insulin stimulation, the level of IRS-1 tyrosine phosphorylation was significantly decreased in tunicamycin-treated XBP-1^{-/-} cells compared with tunicamycin-treated WT controls (FIG. 13H). Insulin-stimulated tyrosine phosphorylation of the insulin receptor was normal in these cells (FIG. 14).

### XBP-1^{+/-} Mice Show Impaired Glucose Homeostasis

Complete XBP-1 deficiency results in embryonic lethality (Reimold et al., Genes Dev. 14:152, 2000; incorporated herein by reference). To investigate the role of XBP-1 in ER stress, insulin sensitivity and systemic glucose metabolism in vivo, we studied Balb/C-XBP-1^{+/-} mice with a null mutation in one XBP-1 allele. We studied mice on the Balb/C genetic background, since this strain exhibits strong resistance to obesity-induced alterations in systemic glucose metabolism. Based on our results with cellular systems, we hypothesized that XBP-1 deficiency would predispose mice to the development of insulin resistance and type 2 diabetes.

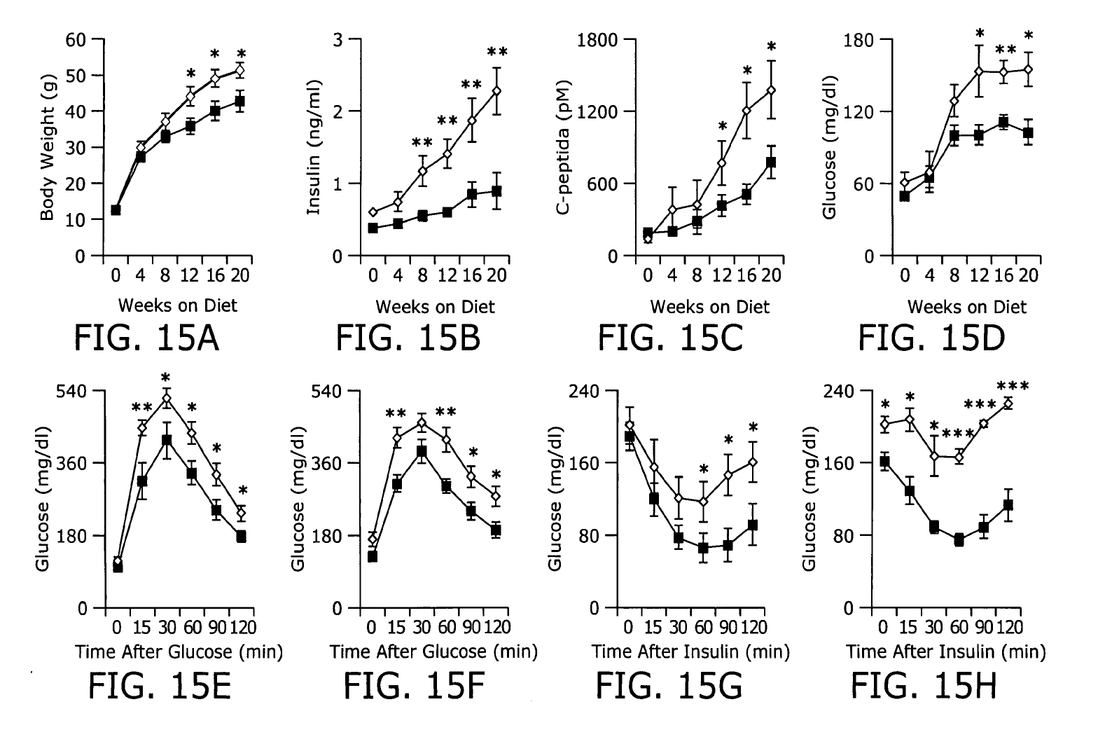

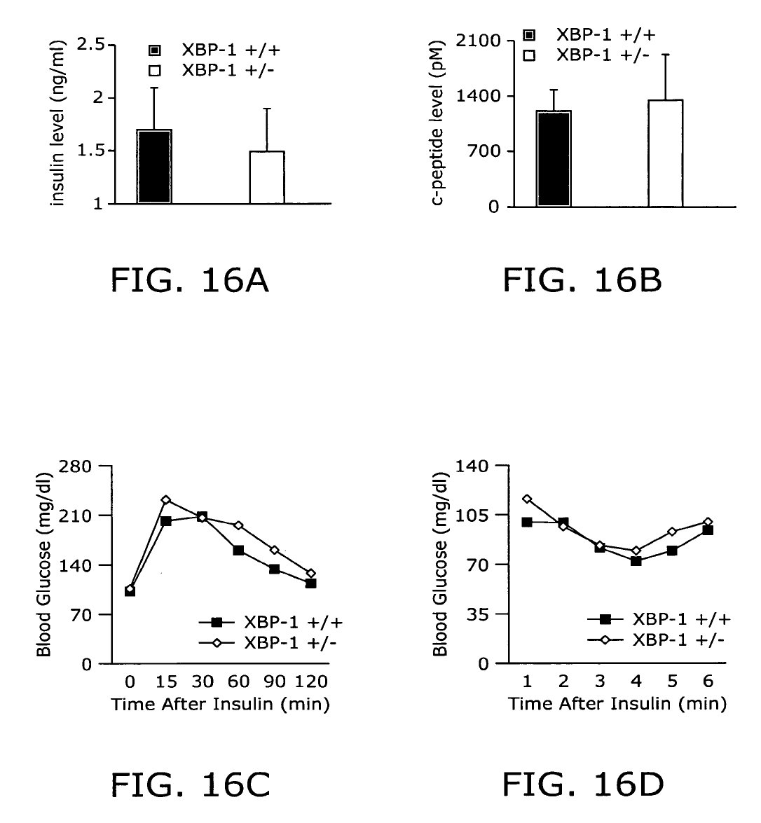

We placed XBP-1^{+/-} mice and their WT littermates on a high fat diet (HFD) at 3 weeks of age. In parallel, control mice of both genotypes were placed on a chow diet. The total body weights of both genotypes were similar on chow diet and until 12 weeks of age on HFD. After this period, the XBP-1^{+/-} animals on HFD exhibited a small but significant increase in body weight (FIG. 15A). Serum levels of leptin, adiponectin and triglycerides did not exhibit any statistically significant differences between the genotypes measured after 16 weeks of HFD (FIG. 16).

On HFD, XBP-1^{+/-} mice developed continuous and progressive hyperinsulinemia evident as early as 4 weeks (FIG. 15B). Insulin levels continued to increase in XBP-1^{+/-} mice for the duration of the experiment. Blood insulin levels in XBP-1^{+/+} mice were significantly lower than those in XBP-1^{+/-} littermates (FIG. 15B). As shown in FIG. 15C, C-peptide levels were also significantly higher in XBP-1^{+/-} animals than in WT controls. Blood glucose levels also began to rise in the XBP-1^{+/-} mice on HFD starting at 8 weeks and remained high until the conclusion of the experiment at 20 weeks (FIG. 15D). This pattern was the same in both fasted (FIG. 15D) and fed (data not shown) states. The rise in blood glucose in the face of hyperinsulinemia in the mice on HFD is a strong indicator of the development of peripheral insulin resistance.

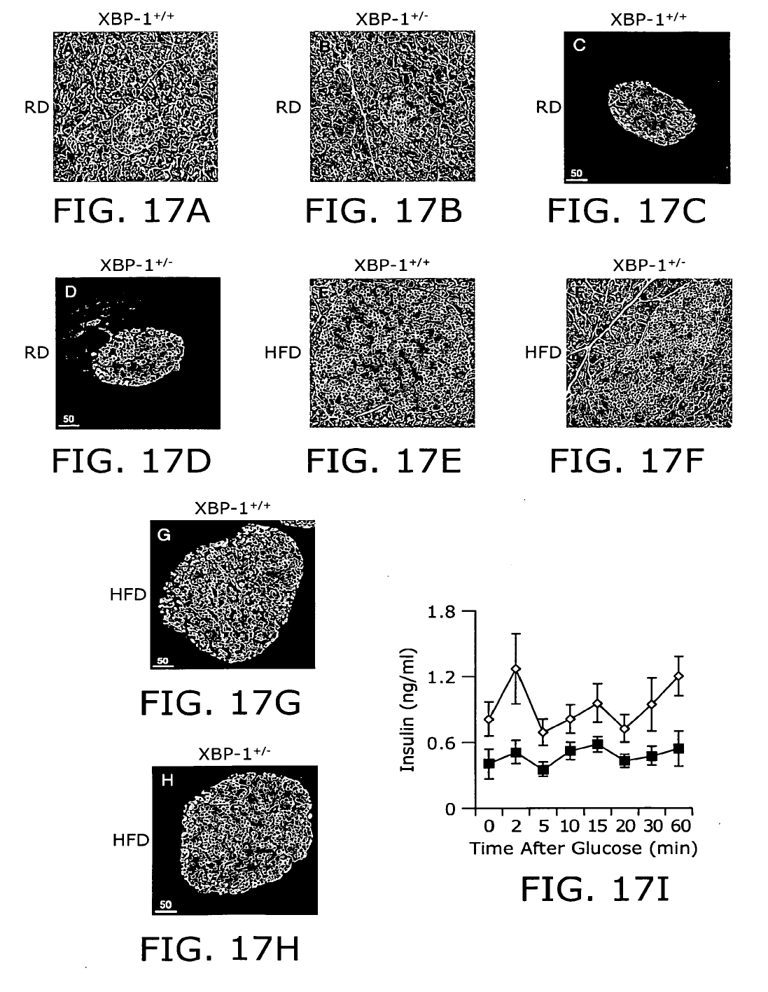

To investigate systemic insulin sensitivity, we performed glucose (GTT) and insulin (ITT) tolerance tests in XBP-1^{+/-} mice and XBP-1^{+/+} controls. Exposure to HFD resulted in significant glucose intolerance in XBP-1^{+/-} mice. Upon glucose challenge after 7 weeks of HFD, XBP-1^{+/-} mice showed significantly higher glucose levels than XBP-1^{+/+} mice (FIG. 15E). This glucose intolerance continued to be evident in XBP-1^{+/-} mice compared with WT mice after 16 weeks on HFD (FIG. 15F). During ITT, the hypoglycemic response to insulin was also significantly lower in XBP-1^{+/-} mice compared with XBP-1^{+/+} littermates at 8 weeks of HFD (FIG. 15G) and this reduced responsiveness continued to be evident after 17 weeks of HFD (FIG. 15H). Examination of islets morphology and function did not reveal significant differences between genotypes (FIG. 17). Hence, loss of an XBP-1 allele predisposes mice to diet-induced insulin resistance and diabetes.

### Increased ER Stress and Impaired Insulin Signaling in XBP-1^{+/-} Mice

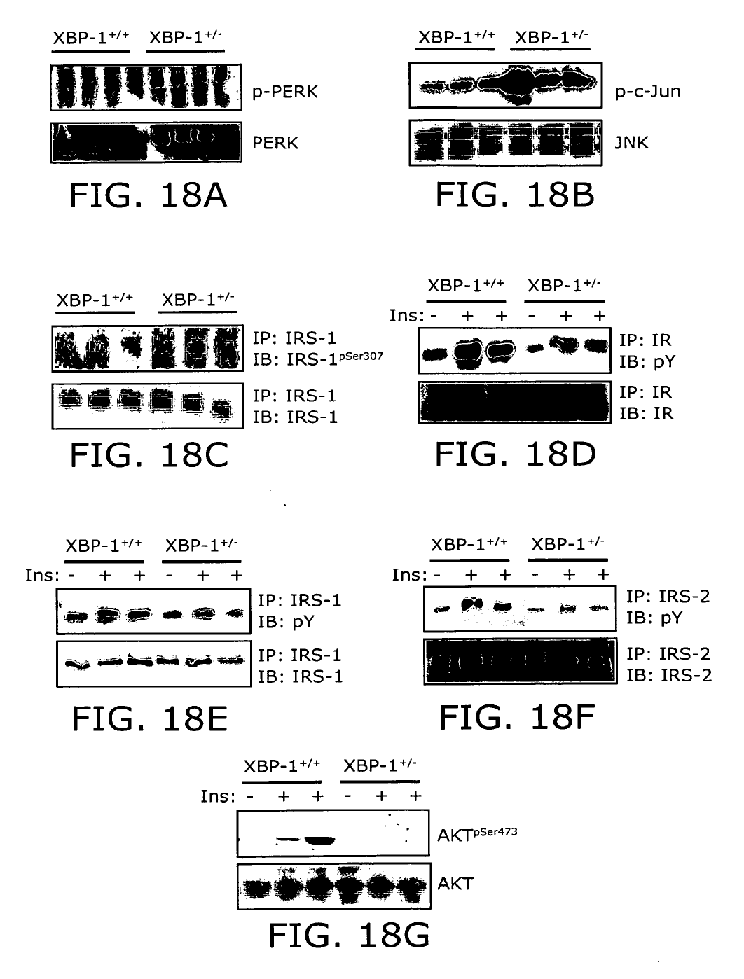

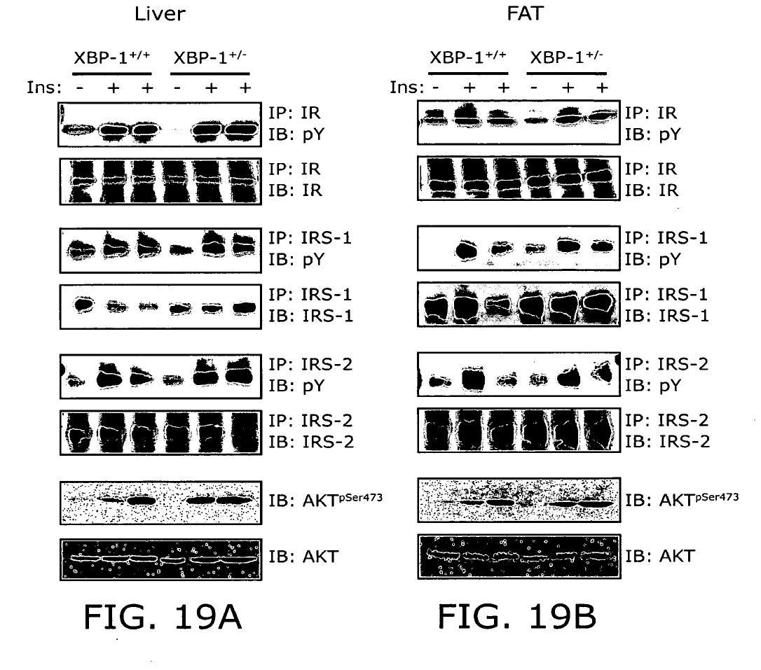

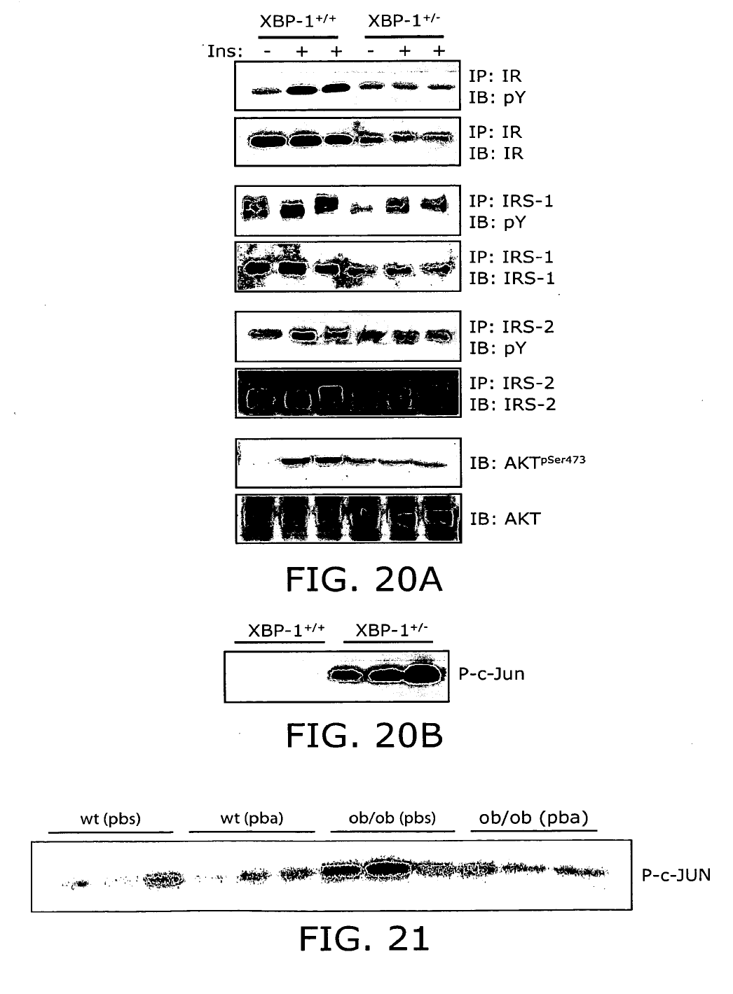

Our experiments with cultured cells demonstrated an increase in ER stress and a decrease in insulin signaling capacity in XBP-1-deficient cells and reversal of these phenotypes upon expression of high levels of XBP-1s. If this mechanism is the basis of the insulin resistance seen in XBP-1^{+/-} mice, these animals should exhibit high levels of ER stress coupled with impaired insulin receptor signaling. To test this, we first evaluated ER stress by examining PERK phosphorylation and JNK activity in the livers of obese XBP-1^{+/-} and WT mice. These experiments revealed an increase in PERK levels and seemingly an increase in liver PERK phosphorylation in obese XBP-1^{+/-} mice compared with WT controls on HFD (FIG. 18A). There was also a significant increase in JNK activity in XBP-1^{+/-} mice compared with WT controls (FIG. 18B). Consistent with these results, serine 307 phosphorylation of IRS-1 was also increased in XBP-1^{+/-} mice compared with WT controls on HFD (FIG. 18C). Finally, we studied in vivo insulin-stimulated insulin receptor-signaling capacity in these mice. There was no detectable difference in any of the insulin receptor signaling components in liver and adipose tissues between genotypes on regular diet (FIG. 19). However, following exposure to HFD, major components of insulin receptor signaling in the liver, including insulin-stimulated IR, IRS-1 and IRS-2 tyrosine- and Akt serine-phosphorylation were all decreased in XBP-1^{+/-} mice compared with WT controls (FIG. 18D-G). A similar suppression of insulin receptor signaling was also evident in the adipose tissues of XBP-1^{+/-} mice compared with XBP-1^{+/+} mice on HFD (FIG. 20). The suppression of IR tyrosine phosphorylation in XBP-1^{+/-} mice differs from the observations made in XBP-1^{-/-} cells where ER stress inhibited insulin action at a post-receptor level. It is likely that this reflects the effects of chronic hyperinsulinemia in vivo on insulin receptors. Hence, our data demonstrate the link between ER stress and insulin action in vivo but are not conclusive in determining the exact locus in insulin receptor signaling pathway that is targeted through this mechanism.

### Discussion

In this study, we identify ER stress as a molecular link between obesity, the deterioration of insulin action and the development of type 2 diabetes.

Our findings point to a fundamental mechanism underlying the molecular sensing of obesity-induced metabolic stress by the ER and inhibition of insulin action that ultimately leads to insulin resistance and type 2 diabetes. We postulate that ER stress underlies the emergence of the stress and inflammatory responses in obesity and the integrated deterioration of systemic glucose homeostasis (Shi et al., Sonenberg, Endocr. Rev. 24:91 (2003); incorporated herein by reference). Our findings differ sharply form earlier work in this area linking ER stress to type 1 diabetes and demonstrate that ER stress is an integral mechanism underlying insulin resistance and type 2 diabetes.

The critical role of ER stress responses in insulin action may represent an evolutionarily conserved mechanism by which stress signals are integrated with metabolic regulatory pathways. Such integration through ER stress would have been advantageous since proper regulation of energy fluxes and suppression of major anabolic pathways such as insulin action might be favorable during acute stress, pathogen invasion and immune responses. However, in the presence of a chronic ER stress such as in obesity, this close link between ER stress and metabolic regulation would lead to development of insulin resistance and eventually, type 2 diabetes. Finally, if the integration of stress signals and metabolic homeostasis through ER stress has a potential positive impact on survival, a highly responsive system would be subject to selection. This selection might be a potential underlying mechanism for the dramatically high prevalence of metabolic diseases in modern times as exposure to excess caloric load would create continuous stress for ER. In terms of therapeutics, our findings suggest that manipulation of the ER stress response offers new opportunities for preventing and treating type 2 diabetes.

### Materials and Methods

Biochemical Reagents: Anti-IRS-1, anti-phospho-IRS-1 (Ser307) and anti-IRS-2 antibodies were from Upstate Biotechnology (Charlottesville, Va.). Antibodies against phosphotyrosine, eIF2α, insulin receptor β subunit, and XBP-1 were from Santa Cruz Biotechnology (Santa Cruz, Calif.). Anti-phospho-PERK, anti-Akt, and anti-phospho-Akt antibodies and c-Jun protein were from Cell Signaling Technology (Beverly, Mass.). Anti-phospho-eIF2α antibody was purchased from Stressgen (Victoria, British Columbia, Canada). Anti-insulin antibody and C-peptide RIA kit were purchased from Linco Research (St. Charles, Mo.). Anti-glucagon antibody was from Zymed (San Francisco, Calif.). PERK antiserum was kindly provided by Dr. David Ron (New York University School of Medicine). Texas red conjugated donkey anti-guinea pig IgG and fluorescein-conjugated (FITC-conjugated) goat anti-rabbit IgG were from Jackson Immuno Research Laboratories (West Grove, Pa.). Thapsigargin, tunicamycin, and JNK inhibitors were from Calbiochem (San Diego, Calif.), Insulin, glucose, and sulindac sulfide were from Sigma (St. Louis, Mo.). The Ultra Sensitive Rat Insulin ELISA kit was from Crystal Chem. Inc. (Downers Grove, Ill.).

Cells: Rat Fao liver cells were cultured with RPMI 1640 (Gibco, Grand Island, N.Y.) containing 10% fetal bovine serum (FBS). At 70-80% confluency, cells were serum depleted for 12 hours before starting the experiments. Reagents including tunicamycin, thapsigargin, and JNK inhibitors were gently added to the culture dishes in the incubator to prevent any environmental stress. JNK inhibitors were added 1 hour before tunicamycin/thapsigargin treatment. The XBP-1^{-/-} mouse embryonic fibroblasts (MEF) (A. H. Lee, N. N. Iwakoshi, L. H. Glimcher, Mol. Cell. Biol. 23:7448 (2003); incorporated herein by reference), IRE-1α^{-/-} MEF cells (kindly provided by Dr. David Ron, New York University School of Medicine), and their wild type controls were cultured in Dulbecco's Modified Eagle Medium (DMEM) (Gibco, Grand Island, N.Y.) containing 10% FBS. A similar protocol was followed for experiments in MEF cells, except that the cells were serum depleted for only 6 hours.

Overexpression of XBP-1s in MEFs: MEF-tet-off cells (BD Biosciences Clontech, Palo Alto, Calif.) were cultured in DMEM with 100 μg/ml G418 and 1 μg/ml doxycycline. The MEF-tet-off cells express exogenous tTA (tetracycline-controlled transactivator) protein, which binds to TRE (tetracycline response element) and activates transcription only in the absence of tetracycline or doxycycline. The cDNA of the spliced form of XBP-1 was ligated into pTRE2hyg2 plasmid (BD Biosciences Clontech, Palo Alto, Calif.). The MEF-tet-off cells were transfected with the TRE2hyg2-XBP-1s plasmid, followed by selection in the presence of 400 μg/ml hygromycin B. Individual clones of stable transfectants were isolated and doxycycline-dependent XBP-1s expression was confirmed by immunoblotting.

Northern Blot Analysis: Total RNA was isolated from mouse liver using Trizol reagent (Invitrogen, Carlsbad, Calif.), separated by 1% agarose gel, and then transferred onto BrightStar Plus nylon membrane (Ambion, Austin, Tex.). GRP78 cDNA probe was prepared from mouse liver total cDNAs by RT-PCR using the following primers: 5′-TGGAGTTCCCCAGATTGAAG-3′ and 5′-CCTGACCCACCTTTTTCTCA-3′. The DNA probes were labeled with ³²P-dCTP using random primed DNA labeling kit (Roche, Indianapolis, Ind.). Hybridization was performed according to the manufacturer's protocol (Ambion, Austin, Tex.) and visualized by Versa Doe Imaging System 3000 (BioRad, Hercules, Calif.).

Protein Extracts From Cells: At the end of each treatment, cells were immediately frozen in liquid nitrogen and kept at −80° C. Protein extracts were prepared with a lysis buffer containing 25 mM Tris-HCl (pH7.4), 2 mM Na₃VO₄, 10 mM NaF, 10 mM Na₄P₂O₇, 1 mM EGTA, 1 mM EDTA, 1% NP-40, 5 μg/ml leupeptin, 5 μg/ml aprotinin, 10 nM okadaic acid, and 1 mM phenylmethylsulfonyl fluoride (PMSF). Immunoprecipitations and immunoblotting experiments were performed with 750 μg and 75 μg total protein, respectively.

Animal Studies and Obesity Models: Adult (10-12 weeks of age) male ob/ob mice and their wild type (WT) littermates were purchased from Jackson Labs. Mice used in the diet-induced obesity model were male C57BL/6. All mice were placed on high fat diet (HFD: 35.5% fat, 20% protein, 32.7% carbohydrates, Bio-Serve) immediately after weaning (at ˜3 weeks of age). The XBP-1^{+/-} and XBP-1^{+/+} mice were on Balb/C genetic background. Insulin and glucose tolerance tests were performed as previously described (Hirosumi et al., Nature 420:333 (2002); incorporated herein by reference). Insulin and C-peptide ELISA were performed according to manufacturer's instructions using mouse standards (Crystal Chem. Inc., Downers Grove, Ill.). Pancreas isolated from 16-week-old mice was fixed in Bouin's fluid and formalin, and paraffin sections were double-stained with guinea pig anti-insulin and rabbit anti-glucagon antibodies. Texas red dye conjugated donkey anti-guinea pig IgG and FITC conjugated Goat anti-rabbit IgG were used as secondary antibodies.

Insulin Infusion and Tissue Protein Extraction: Insulin was injected through the portal vein as previously described (Uysal et al., Nature 389:610 (1997); Hirosumi et al., Nature 420:333 (2002); each of which is incorporated herein by reference). Three minutes after insulin infusion, liver was removed and frozen in liquid nitrogen and kept at −80° C. until processing. For protein extraction, liver tissue (˜0.3 g) was placed in 10 ml of lysis buffer containing 25 mM Tris-HCl (pH7.4), 10 mM Na₃VO₄, 100 mM NaF, 50 mM Na₄P₂O₇, 10 mM EGTA, 10 mM EDTA, 1% NP-40, 5 μg/ml leupeptin, 5 μg/ml aprotinin, 10 nM okadaic acid, and 2 mM PMSF. After homogenization on ice, the tissue lysate was centrifuged at 4,000 rpm for 15 minutes at 4° C. followed by 55,000 rpm for 1 hour at 4° C. One milligram of total tissue protein was used for immunoprecipitation and subsequent immunoblotting, whereas 100-150 μg total tissue protein was used for direct immunoblotting (Hirosumi et al., Nature 420:333 (2002); incorporated herein by reference).

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

### APPENDIX A

### Drugs for Used in Combination with Er Stress Modulators

### Anti-Diabetic Drugs

### Peroxisome Proliferator-Activated Receptor-Gamma Agonists

Rosiglitazone (GlaxoSmithKline's Avandia)

Pioglitazone (Takeda/Eli Lilly's Actos)

Isaglitazone (Mitsubishi Pharma)

rivoglitazone (Sankyo's CS-011)

T-131 (Amgen)

MBX-102 (Metabolex)

R-483 (Roche Chugai)

CLX-0921 (Calyx)

### Biguanides

Metformin (Bristol-Myers Squibb's Glucophage, generics, Bristol-Myers Squibb's Glucophage XR)

### Sulfonylureas

Glimepiride (Aventis's Amaryl)
Glyburide/Glibenclamide (Aventis's Diabeta, Pharmacia's Micronase, generics, Pharmacia' s Glynase)
Glipizide (Pfizer's Glucotrol, generics, Pfizer's Glucotrol XL)
Gliclazide (Servier's Diamicron, Molteni & C.F. LLI Alitti's Diabrezide, Irex-Synthélabo's Glycemirex, Dainippon's Glimicron, generics)

### Combination Agents

Metformin/Glyburide (Bristol-Myers Squibb's Glucovance, Hoechst's Suguan M)

Metformin/Rosiglitazone (GlaxoSmithKline's Avandamet)

Metformin/Glipizide (Bristol-Myers Squibb's Metaglip)

### Meglitinides

Repaglinide (Novo Nordisk's Prandin/NovoNorm)

Nateglinide (Novartis's Starlix/Starsis)

### Alpha-Glucosidase Inhibitors

Acarbose (Bayer's Precose/Glucobay, generics)

Miglitol (Pharmacia's Glyset)

Voglibose (Takeda's Basen)

### Insulin and Insulin Analogues

Insulin lispro (Eli Lilly's Humalog)

Insulin glargine (Aventis's Lantus)

Exubera (Nektar/Pfizer/Aventis)

AERx Insulin Diabetes Management System (Aradigm/Novo Nordisk)

AIR inhaled insulin (Eli Lilly/Alkermes)

Oralin (Generex)

Insulin detemir (NN-304) (Novo Nordisk)

Insulin glulisine (Aventis)

### Dual PPAR Agonists and PPAR Pan Agonists

BMS-298585 (Bristol-Myers Squibb/Merck)

Tesaglitazar (AstraZeneca's Galida)

muraglitazar (BMS-Merck and Co)

naveglitazar (Lilly-Ligand's LY-818)

TAK-559 (Takeda)

netoglitazone (Mitsubishi)

GW-677594 (GSK)

AVE-0847 (Aventis)

LY-929 (Lilly-Ligand)

ONO-5129 (ONO)

### Glucagon-Like Peptide-1 (GLP-1) Analogues and Agonists

Exenatide (AC-2993) (Eli Lilly/Amylin Pharmaceuticals)

Exenatide LAR (AC-2993 LAR) (Amylin Pharmaceuticals/Alkermes/Eli Lilly)

Liraglutide (insulinotropin/NN-2211) (Scios/Novo Nordisk)

CJC-1131 (ConjuChem)

AVE-0010 (Aventis-Zealand)

BIM-51077 (Roche-Teijin-Ipsen)

NN-2501 (Novo Nordisk)

SUN-E7001 (Daiichi Suntory—Sankyo)

### Dipeptidyl Peptidase IV (DPP-IV) Inhibitors

LAF-237 (Novartis)

MK-431 (Merck and Co)

PSN-9301 (Probiodrug Prosidion)

815541 (GlaxoSmithKline-Tanabe)

823093 (GlaxoSmithKline)

825964 (GlaxoSmithKline)

BMS-477118 (BMS)

### Pancreatic Lipase Inhibitors

Orlistat (Roche Holding)

### Sodium Glucose co Transporter (SGLT) Inhibitors

T-1095 (Tanabe-J&J)

AVE-2268 (Aventis)

869682 (GlaxoSmithKline-Kissei)

### Amylin Analog

pramlintide (Amylin's Symlin)

Other Drugs from the PDR:

### Indication=Hyperglycemia

### Actos Tablets (Takeda)

Pioglitazone Hydrochloride

### Amaryl Tablets (Sanofi-Aventis)

Glimepiride

### Apidra Injection (Sanofi-Aventis)

Insulin Glulisine

### Avandamet Tablets (GlaxoSmithKline)

Metformin Hydrochloride, Rosiglitazone Maleate

### Avandia Tablets (GlaxoSmithKline)

Rosiglitazone Maleate

### DiaBeta Tablets (Sanofi-Aventis)

Glyburide

### Fortamet Extended-Release Tablets (Andrx Labs)

Metformin Hydrochloride

### Glucotrol XL Extended Release Tablets (Pfizer)

Glipizide

### Metaglip Tablets (Bristol-Myers Squibb)

Glipizide, Metformin Hydrochloride

### Prandin Tablets (0.5, 1, and 2 mg) (Novo Nordisk)

Repaglinide

### Precose Tablets (Bayer)

Acarbose

### Starlix Tablets (Novartis)

Nateglinide

Other Drugs from the PDR:

### Indication=Insulin Dependent Diabetes

### Humalog-Pen (Lilly)

Insulin Lispro, Human

### Humalog Mix 75/25-Pen (Lilly)

Insulin Lispro Protamine, Human, Insulin Lispro, Human

### Humulin 50/50, 100 Units (Lilly)

Insulin, Human Regular and Human NPH Mixture

### Humulin 70/30 Pen (Lilly)

Insulin, Human Regular and Human NPH Mixture

### Humulin L, 100 Units (Lilly)

Insulin, Human, Zinc Suspension

### Humulin N, 100 Units (Lilly)

Insulin, Human NPH

### Humulin R (U-500) (Lilly)

Insulin, Human Regular

### Humulin R, 100 Units (Lilly)

Insulin, Human Regular

### Humulin U, 100 Units (Lilly)

Insulin, Human, Zinc Suspension

### Humulin N Pen (Lilly)

Insulin, Human NPH

### Innovo (Novo Nordisk)

Device

### Lantus Injection (Sanofi-Aventis)

Insulin glargine

### Novolin 70/30 Human Insulin 10 ml Vials (Novo Nordisk)

Insulin, Human Regular and Human NPH Mixture

### Novolin 70/30 PenFill 3 ml Cartridges (Novo Nordisk)

Insulin, Human Regular and Human NPH Mixture

### Novolin N Human Insulin 10 ml Vials (Novo Nordisk)

Insulin, Human NPH

### Novolin N PenFill 3 ml Cartridges (Novo Nordisk)

Insulin, Human NPH

### Novolin R Human Insulin 10 ml Vials (Novo Nordisk)

Insulin, Human Regular

### Novolin R PenFill 1.5 ml Cartridges (Novo Nordisk)

Insulin, Human Regular

### Novolin R PenFill 3 ml Cartridges (Novo Nordisk)

Insulin, Human Regular

### NovoLog Injection (Novo Nordisk)

Insulin Aspart, Human Regular

### NovoLog Mix 70/30 (Novo Nordisk)

Insulin Aspart Protamine, Human, Insulin Aspart, Human

Other Drugs from the PDR:

### Indication=Non-Insulin Dependent Diabetes

### Actos Tablets (Takeda)

Pioglitazone Hydrochloride

### Amaryl Tablets (Sanofi-Aventis)

Glimepiride

### Avandamet Tablets (GlaxoSmithKline)

Metformin Hydrochloride, Rosiglitazone Maleate

### Avandia Tablets (GlaxoSmithKline)

Rosiglitazone Maleate

### DiaBeta Tablets (Sanofi-Aventis)

Glyburide

### Glucotrol XL Extended Release Tablets (Pfizer)

Glipizide

### Lantus Injection (Sanofi-Aventis)

Insulin glargine

### Metaglip Tablets (Bristol-Myers Squibb)

Glipizide, Metformin Hydrochloride

### Prandin Tablets (0.5, 1, and 2 mg) (Novo Nordisk)

Repaglinide

### Precose Tablets (Bayer)

Acarbose

### Starlix Tablets (Novartis)

Nateglinide

Other Drugs from the PDR:

### Indication=Type 1 Diabetes

### Lantus Injection (Sanofi-Aventis)

Insulin glargine

### Novolin 70/30 InnoLet (Novo Nordisk)

Insulin, Human Regular and Human NPH Mixture

### Novolin N InnoLet (Novo Nordisk)

Insulin, Human NPH

### Novolin R InnoLet (Novo Nordisk)

Insulin, Human NPH

Other Drugs from the PDR:

### Indication=Type 2 Diabetes

### Fortamet Extended-Release Tablets (Andrx Labs)

Metformin Hydrochloride

### Lantus Injection (Sanofi-Aventis)

Insulin glargine

### Prandin Tablets (0.5, 1, and 2 mg) (Novo Nordisk)

Repaglinide

anti-obesity drugs

### Pancreatic Lipase Inhibitors

Orlistat (Roche's Xenical, Roche Nippon's Xenical)

### Serotonin and Norepinephrine Reuptake Inhibitors

Sibutramine (Abbott/Knoll's Meridia, AstraZeneca's Reductil, Eisai's Reductil)

### Noradrenergic Anorectic Agents

Phentermine (GlaxoSmithKline's Fastin, Medeva's Ionamin)

Mazindol (Wyeth-Ayerst's Mazanor, Novartis's Sanorex)

### Peripherally Acting Agents

ATL-962 (Alizyme)

HMR-1426 (Aventis)

GI-181771 (GlaxoSmithKline)

### Centrally Acting Agents

Recombinant human ciliary neurotrophic factor (Axokine) (Regeneron)

Rimonabant (SR-141716) (Sanofi-Synthélabo)

BVT-933 (GlaxoSmithKline/Biovitrum)

Bupropion SR (GlaxoSmithKline)

P-57 (Phytopharm)

### Thermogenic Agents

TAK-677 (AJ-9677) (Dainippon/Takeda)

### Cannabinoid CB1 Antagonist

Acomplia (Sanofi-Aventis)

SLV319 (Solvay)

### Ciliary Neurotrophic Factor (CNTF) Agonists

Axokine (Regeneron)

### Other Anti-Obesity Drugs

1426 (Sanofi-Aventis)

1954 (Sanofi-Aventis)

c-2624 (Merck & Co)

c-5093 (Merck & Co)

T71 (Tularik)

### Cholecystokinin (CCK) Agonist

GI 181771 (GSK)

### Lipid Metabolism Modulator

AOD9604 (Monash University/Metabolic Pharmaceuticals)

### Lipase Inhibitor

ATL962 (Alizyme, Takeda)

### Glucagon-Like Peptide 1 Agonist

AC 137 (Amylin)

### Leptin Agonist

Second generation leptin (Amgen)

### Beta-3 Adrenergic Agonist

SR58611 (Sanofi-Aventis)

CP 331684 (Pfizer)

LY 377604 (Eli Lilly)

n5984 (Nisshin Kyorin Pharmaceutical)

### Peptide Hormone

peptide YY [3-36] (Nastech)

### CNS Modulator

s2367 (Shionogi & Co Ltd)

### Neutrotrophic Factor

Peg axokine (Regeneron)

### 5HT2C Serotonin Receptor Agonist

APD356 (Arena Pharmaceutical)

### Peptide YY [3-36]

AC162352 (Amylin)

Other Drugs from the PPM:

### Indication=Obesity

### Adipex-P Capsules (Gate)

Phentermine Hydrochloride

### Adipex-P Tablets (Gate)

Phentermine Hydrochloride

### Desoxyn Tablets, USP (Ovation)

Methamphetamine Hydrochloride

### Ionamin Capsules (Celltech)

Phentermine Resin

### Meridia Capsules (Abbott)

Sibutramine Hydrochloride Monohydrate

### Xenical Capsules (Roche Laboratories)

Orlistat

Other Drugs from the PDR:

### Prescribing Category=Appetite Suppressant

### Adipex-P Capsules (Gate)

Phentermine Hydrochloride

### Adipex-P Tablets (Gate)

Phentermine Hydrochloride

### Desoxyn Tablets, USP (Ovation)

Methamphetamine Hydrochloride

### Ionamin Capsules (Celltech)

Phentermine Resin

### Meridia Capsules (Abbott)

Sibutramine Hydrochloride Monohydrate

### Anti-Atherosclerosis Drugs

### HMG-CoA Reductase Inhibitors (Statins)

Atorvastatin (Warner-Lambert/Pfizer's Lipitor)

Pravastatin (Bristol-Myers Squibb's Pravachol/Sankyo's Mevalotin)

Simvastatin (Merck & Co.'s Zocor)

Lovastatin (Merck & Co.'s Mevacor)

Fluvastatin (Novartis's Lescol)

Cerivastatina (Bayer's Lipobay/GlaxoSmithKline's Baycol)
Rosuvastatin (AstraZeneca's Crestor)
Pitivastatin (itavastatin/risivastatin) (Nissan/Kowa/Sankyo/Novartis)

### Fibrates

Bezafibrate (Boehringer Mannheim/Roche's Bezalip, Kissei's Bezatol)

Clofibrate (Wyeth-Ayerst's Atromid-S, generics)

Fenofibrate (Fournier's Lipidil, Abbott's Tricor, Takeda's Lipantil, generics)

Gemfibrozil (Pfizer's Lopid, generics)

### Bile Acid Sequestrants

Cholestyramine Bristol-Myers Squibb's Questran and Questran Light, generics

Colestipol Pharmacia's Colestid

### Niacin

Niacin—immediate release (Aventis's Nicobid, Upsher-Smith's Niacor, Aventis's Nicolar, Sanwakagaku's Perycit, generics
Niacin—extended release (Kos Pharmaceuticals' Niaspan, Upsher-Smith's Slo-Niacin)

### Antiplatelet Agents

Aspirin (Bayer's Aspirin, generics)

Clopidogrel (Sanofi-Synthélabo/Bristol-Myers Squibb's Plavix)

Ticlopidine (Sanofi-Synthélabo's Ticlid, Daiichi's Panaldine, generics)

### Angiotensin-Converting Enzyme Inhibitors

Ramipril (Aventis's Altace)

Enalapril (Merck & Co.'s Vasotec)

### Angiotensin II Receptor Antagonists

Losartan potassium (Merck & Co.'s Cozaar)

### Acyl CoA Cholesterol Acetyltransferase (ACAT) Inhibitors

Avasimibe (Pfizer)

Eflucimibe (BioMérieux Pierre Fabre/Eli Lilly)

CS-505 (Sankyo and Kyoto)

SMP-797 (Sumito)

### Cholesterol Absorption Inhibitors

Ezetimibe (Schering-Plough/Merck & Co.)

Pamaqueside (Pfizer)

### Cholesterol Ester Transfer Protein (CETP) Inhibitors

CP-529414 (Pfizer)

JTT-705 (Japan Tobacco)

CETi-1 (Avant Immunotherapeutics)

### Microsomal Triglyceride Transfer Protein (MTTP) Inhibitors

Implitapide (Bayer)

R-103757 (Janssen)

### Other Cholesterol Modulators

NO-1886 (Otsuka/TAP Pharmaceutical)

CI-1027 (Pfizer)

WAY-135433 (Wyeth-Ayerst)

### Bile Acid Modulators

GT102-279 (GelTex/Sankyo)

HBS-107 (Hisamitsu/Banyu)

### Peroxisome Proliferation Activated Receptor (PPAR) Agonists

Tesaglitazar (AZ-242) (AstraZeneca)

Netoglitazone (MCC-555) (Mitsubishi/Johnson & Johnson)

GW-409544 (Ligand Pharmaceuticals/GlaxoSmithKline)

GW-501516 (Ligand Pharmaceuticals/GlaxoSmithKline)

### Gene-Based Therapies

AdGVVEGF121.10 (GenVec)
ApoA1 (UCS Pharma/Groupe Fournier)
EG-004 (Trinam) (Ark Therapeutics)
ATP-binding cassette transporter-A1 (ABCA1) (CV Therapeutics/Incyte, Aventis, Xenon)

### Composite Vascular Protectants

AGI-1067 (Atherogenics)

### Other Anti-Atherosclerotic Agents

BO-653 (Chugai Pharmaceuticals)

### Glycoprotein IIb/IIIa Inhibitors

Roxifiban (Bristol-Myers Squibb)

Gantofiban (Yamanouchi)

Cromafiban (Millennium Pharmaceuticals)

### Aspirin and Aspirin-Like Compounds

Asacard (slow-release aspirin) (Pharmacia)

Pamicogrel (Kanebo/Angelini Ricerche/CEPA)

### Combination Therapies

Advicor (niacin/lovastatin) (Kos Pharmaceuticals)

Amlodipine/atorvastatin (Pfizer)

Simvastatin/ezetimibe (Merck & Co./Schering-Plough)

### IBAT Inhibitors

S-8921 (Shionogi)

### Squalene Synthase Inhibitors

BMS-188494 I(Bristol-Myers Squibb)

CP-210172 (Pfizer)

CP-295697 (Pfizer)

CP-294838 (Pfizer)

### Monocyte Chemoattractant Protein (MCP)-1 Inhibitors

RS-504393 (Roche Bioscience)

Other MCP-1 inhibitors (GlaxoSmithKline, Teijin, and Bristol-Myers Squibb)

Other Drugs from the PDR:

### Indication=Hypercholesterolemia

### Advicor Tablets (Kos)

Lovastatin, Niacin

### Altoprev Extended-Release Tablets (Andrx Labs)

Lovastatin

### Caduet Tablets (Pfizer)

Amlodipine Besylate, Atorvastatin Calcium

### Crestor Tablets (AstraZeneca)

Rosuvastatin Calcium

### Lescol Capsules (Novartis)

Fluvastatin Sodium

### Lescol Capsules (Reliant)

Fluvastatin Sodium

### Lescol XL Tablets (Novartis)

Fluvastatin Sodium

### Lescol XL Tablets (Reliant)

Fluvastatin Sodium

### Lipitor Tablets (Parke-Davis)

Atorvastatin Calcium

### Lofibra Capsules (Gate)

Fenofibrate

### Mevacor Tablets (Merck)

Lovastatin

### Niaspan Extended-Release Tablets (Kos)

Niacin

### Pravachol Tablets (Bristol-Myers Squibb)

Pravastatin Sodium

### Tricor Tablets (Abbott)

Fenofibrate

### Vytorin 10/10 Tablets (Merck/Schering Plough)

Ezetimibe, Simvastatin

### Vytorin 10/10 Tablets (Schering)

Ezetimibe, Simvastatin

### Vytorin 10/20 Tablets (Merck/Schering Plough)

Ezetimibe, Simvastatin

### Vytorin 10/20 Tablets (Schering)

Ezetimibe, Simvastatin

### Vytorin 10/40 Tablets (Merck/Schering Plough)

Ezetimibe, Simvastatin

### Vytorin 10/40 Tablets (Schering)

Ezetimibe, Simvastatin

### Vytorin 10/80 Tablets (Merck/Schering Plough)

Ezetimibe, Simvastatin

### Vytorin 10/80 Tablets (Schering)

Ezetimibe, Simvastatin

### WelChol Tablets (Sankyo)

Colesevelam Hydrochloride

### Zetia Tablets (Schering)

Ezetimibe

### Zetia Tablets (Merck/Schering Plough)

Ezetimibe

### Zocor Tablets (Merck)

Simvastatin

### Anti-Dyslipidemia Drugs

### HMG-CoA Reductase Inhibitors

Atorvastatin (Pfizer's Lipitor/Tahor/Sortis/Torvast/Cardyl)
Simvastatin (Merck's Zocor/Sinvacor, Boehringer Ingelheim's Denan, Banyu's Lipovas)
Pravastatin (Bristol-Myers Squibb's Pravachol, Sankyo's Mevalotin/Sanaprav)
Fluvastatin (Novartis's Lescol/Locol/Lochol, Fujisawa's Cranoc, Solvay's Digaril)
Lovastatin (Merck's Mevacor/Mevinacor, Bexal's Lovastatina, Cepa; Schwarz Pharma's Liposcler)
Rosuvastatin (AstraZeneca's Crestar)
Pitavastatin (Nissan Chemical, Kowa Kogyo, Sankyo, and Novartis)

### HMG-CoA Reductase Inhibitor Combination Therapies

Simvastatin/ezetimibe (Merck and Schering-Plough)

### Fibrates

Fenofibrate (Abbott's Tricor, Fournier's Lipidil/Lipantil)

Bezafibrate (Roche's Béfizal/Cedur/Bezalip, Kissei's Bezatol, generics)

Gemfibrozil (Pfizer's Lopid/Lipur, generics)

Clofibrate (Wyeth's Atromid-S, generics)

Ciprofibrate (Sanofi-Synthélabo's Modalim)

### Bile Acid Sequestrants

Colestyramine (Bristol-Myers Squibb's Questran)

Colestipol (Pfizer's Colestid)

Colesevelam (Genzyme/Sankyo's Welchol)

### Cholesterol Absorption Inhibitors

Ezetimibe (Merck and Schering-Plough's Zetia)

Pamaqueside (Pfizer)

### Nicotinic Acid Derivatives

Nicotinic acid (Kos's Niaspan, Yamanouchi's Nyclin)

### Acyl-CoA Cholesterol Acyltransferase Inhibitors

Avasimibe (Pfizer)

Eflucimibe (Eli Lilly)

### Cholesteryl Ester Transfer Protein Inhibitors

Torcetrapib (Pfizer)

JTT-705 (Japan Tobacco)

CETi-1 (Avant Immunotherapeutics)

### Microsomal Triglyceride Transfer Protein Inhibitors

Implitapide (Bayer)

CP-346086 (Pfizer)

### Peroxisome Proliferation Activated Receptor Agonists

GW-501516 (Ligand Pharmaceuticals and GlaxoSmithKline)

Tesaglitazar (AstraZeneca)

LY-929 (Ligand Pharmaceuticals and Eli Lilly)

LY-465608 (Ligand Pharmaceuticals and Eli Lilly)

LY-518674 (Ligand Pharmaceuticals and Eli Lilly)

MK-767 (Merck and Kyorin)

### Squalene Synthase Inhibitors

TAK-475 (Takeda)

### Other New Approaches

MBX-102 (Metabolex)

NO-1886 (Otsuka)

Gemeabene (Pfizer)

### Liver X Receptor Agonists

GW-3965 (GlaxoSmithKline)

TU-0901317 (Tularik)

### Bile Acid Modulators

BTG-511 (British Technology Group)

HBS-107 (Hisamitsu and Banyu)

BARI-1453 (Aventis)

S-8921 (Shionogi)

SD-5613 (Pfizer)

AZD-7806 (AstraZeneca)

Other Drugs from the PDR:

### Indication=Hypercholesterolemia

### Advicor Tablets (Kos)

Lovastatin, Niacin

### Altoprev Extended-Release Tablets (Andrx Labs)

Lovastatin

### Caduet Tablets (Pfizer)

Amlodipine Besylate, Atorvastatin Calcium

### Crestor Tablets (AstraZeneca)

Rosuvastatin Calcium

### Lescol Capsules (Novartis)

Fluvastatin Sodium

### Lescol Capsules (Reliant)

Fluvastatin Sodium

### Lescol XL Tablets (Novartis)

Fluvastatin Sodium

### Lescol XL Tablets (Reliant)

Fluvastatin Sodium

### Lipitor Tablets (Parke-Davis)

Atorvastatin Calcium

### Lofibra Capsules (Gate)

Fenofibrate

### Mevacor Tablets (Merck)

Lovastatin

### Niaspan Extended-Release Tablets (Kos)

Niacin

### Pravachol Tablets (Bristol-Myers Squibb)

Pravastatin Sodium

### Tricor Tablets (Abbott)

Fenofibrate

### Vytorin 10/10 Tablets (Merck/Schering Plough)

Ezetimibe, Simvastatin

### Vytorin 10/10 Tablets (Schering)

Ezetimibe, Simvastatin

### Vytorin 10/20 Tablets (Merck/Schering Plough)

Ezetimibe, Simvastatin

### Vytorin 10/20 Tablets (Schering)

Ezetimibe, Simvastatin

### Vytorin 10/40 Tablets (Merck/Schering Plough)

Ezetimibe, Simvastatin

### Vytorin 10/40 Tablets (Schering)

Ezetimibe, Simvastatin

### Vytorin 10/80 Tablets (Merck/Schering Plough)

Ezetimibe, Simvastatin

### Vytorin 10/80 Tablets (Schering)

Ezetimibe, Simvastatin

### WelChol Tablets (Sankyo)

Colesevelam Hydrochloride

### Zetia Tablets (Schering)

Ezetimibe

### Zetia Tablets (Merck/Schering Plough)

Ezetimibe

### Zocor Tablets (Merck)

Simvastatin

### Anti-Hypertension Drugs

### Diuretics

Chlorthalidone (Alliance's Hygotron, generics)
Metolazone (Generics)
Indapamide (Servier's Natrilix/Tertensif, Aventis's Lozol, Merck's Indapamide, generics)
Bumetanide (Leo's Burinex, Sankyo's Lunetoron, Roche's Bumex, generics)
Ethacrynic acid (Merck & Co.'s Edecrin)
Furosemide (Aventis's Lasilix/Lasix/Eutensin/Seguril, generics)
Torsemide (Roche's Demadex, generics)
Amiloride hydrochloride (Merck & Co.'s Midamor, generics)
Spironolactone (Pharmacia's Spirolang/Aldactone, Mylan's Spironolactone, generics)
Triamterene (GlaxoSmithKline's Dyrenium, Goldshield's Dytac, Isei's Triamterene/Triteren)

### Alpha Blockers

Doxazosin mesylate (Pfizer's Zoxan/Cardura/Cardenalin, Hexyl's Doxacor, generics)
Prazosin hydrochloride (Pfizer's Minipress, generics)
Terazosin hydrochloride (Abbott's Hytrin/Flotrin/Heitrin/Itrin/Deflox, Mitsubishi Welpharma's Vasomet, generics)

### Beta Blockers

Acebutolol (Bayer's Prent, Wyeth's Sectral, Aventis's Acetanol, generics)
Atenolol (AstraZeneca/Sumitomo's Tenormin, generics)
Betaxolol (Sanofi-Synthélabo's Kerlone, generics)
Bisoprolol fumarate (Merck KGaA's Cardicor, Lipha Sante's Detensiel/Cardensiel, Lederle's Monocor/Zebeta, generics)
Carteolol Hydrochloride (Lipha Sante/Otsuka's Mikelan, Abbott's Cartrol)
Metoprolol tartrate (Novartis's Lopressor/Prelis, AstraZeneca's Seloken/Betaloc, generics)
Metoprolol succinate (AstraZeneca's Toprol-XL Herz-mite, generics)
Nadolol (Bristol-Myers Squibb/Sanofi-Synthélabo's Corgard, generics)
Penbutolol sulfate (Schwarz Pharma's Levatol, Wolff/Aventis's BetaPressin)
Pindolol (Novartis's Visken, generics)
Propranolol hydrochloride (AstraZeneca's Inderal/Dociton, Wyeth's Inderal LA, generics)
Timolol maleate (Merck & Co.'s Blocadren, generics)
Carvedilol (Roche's Coreg/Dilatrend/Coropres/Eucardic, GlaxoSmithKline's Coreg)

### Calcium-Channel Blockers

Amlodipine besylate (Pfizer's Amlor/Istin/Norvasc)
Felodipine (AstraZeneca's Flodil/Modip/Plendil/Splendil)
Isradipine (Novartis's Lomir/Prescal)
Nicardipine (Roche/Yamanouchi's Cardene, generics)
Nifedipine (Bayer's Adalat, generics)
Nisoldipine (AstraZeneca's Sular, Bayer's Syscor MR/Baymycard)
Diltiazem hydrochloride (Sanofi-Synthélabo's Tildiem Angizem 60, Pfizer's Dinisor, Generics)
Verapamil Hydrochloride (Knoll's Isoptin Press/Manidon RetardlSecuron, Pharmacia's Calan/Covera, generics)
Azelnidipine (Sankyo/UBE)
Pranidipine (Otsuka)
Graded diltiazem formulation (Biovail)
(S)-amlodipine (Sepracor/Emcure)
clevidipine (AstraZeneca/The Medicines Company)

### Angiotensin-Converting Enzyme Inhibitors

Benazepril hydrochloride (Novartis's Cibacen/Lotensin)
Captopril (Bristol-Myers Squibb's Lopril/Lopirin/Capoten/Acepress, Sanofi-Synthélabo's Alopresin, generics)
Enalapril maleate (Merck & Co.'s Vasotec, Banyu's Renivoce, generics)
Fosinopril sodium (Bristol-Myers Squibb's Fosinorm/Tensogard/Fosinil/Staril/Monopril)
Lisinopril (Merck & Co.'s Prinivil, AstraZeneca's Zestril)
Moexipril (Schwarz's Moex/Fempress/Univasc)
Perindopril (Servier's Conversyl)
Quinapril Hydrochloride (Pfizer's Accupril/Acuitel/Accuprin/Acuprel, Sanofi-Synthélabo's Korec, generics)
Ramipril (Aventis's Altace)
Trandolapril (Abbott's Gopten/Mavik)

### Angiotensin II Receptor Antagonists

Losartan (Merck & Co.'s Cozaar)

Valsartan (Novartis's Tareg/Diovan, Aventis's Nisis)

Irbesartan (Bristol-Myers Squibb/Sanofi-Synthélabo's Aprovel/Karvea)

Candesartan (AstraZeneca's Atacand/Ratacand/Ainias, Takeda's Blopress)

Telmisartan (Boehringer Ingelheim's Micardis)

Eprosartan (Solvay's Teveten)

Olmesartan (Sankyo/Recordati/Menarini/Forest/Kowa)

YM-358 (Yamanouchi)

### Combination Therapies

Benazepril hydrochloride/hydrochlorothiazide (Novartis's Cibadrex/Lotensin HCT)
Captopril/hydrochlorothiazide (Bristol-Myers Squibb's Capozide/Ecazide)
Enalapril maleate/hydrochlorothiazide (Merck & Co.'s Vasorectic/Co Renitec/Innozide, AstraZeneca's Lexxel, generics)
Lisinopril/Hydrochlorothiazide (Merck & Co.'s Prinzide, AstraZeneca's Zestorectic)
Losartan/hydrochlorothiazide (Merck & Co.'s Hyzaar)
Atenolol/Chlorthalidone (AstraZeneca's Tenoretic, generics)
Bisoprolol fumarate/Hydrochlorothiazide (Lederle's Ziac, generics)
Metoprolol tartrate/hydrochlorothiazide (Novartis's Lopressor HCT, Pharmacia's Selopresin/Selozide)
Amlodipine besylate/benazepril hydrochloride (Pfizer's Norvase, Novartis's Lotrel)
Felodipine/enalapril maleate (AstraZeneca's Lexxel)
Verapamil hydrochloride/trandolapril (Knoll/Abbott's Tarka, Aventis's Udramil)
Lercanidipine and enalapril (Recordati/Pierre Fabre)
Olmesartan/hydrochlorothiazide (Sankyo)
Eprosartan/hydrochlorothiazide (Unimed)
Amlodipine besylate/atorvastatin (Pfizer)
Nitrendipine/enalapril (Vita Invest)

### Vasopeptidase Inhibitors

Omapatrilat (Bristol-Myers Squibb)

Gemopatrilat (Bristol-Myers Squibb)

Fasidotril (Eli Lilly)

sampatrilat (Pfizer/Shire)

AVE 7688 (Aventis)

M100240 (Aventis)

Z13752A (Zambon/GSK)

796406 (Zambon/GSK)

### Dual Neutral Endopeptidase and Endothelin Converting Enzyme (NEP/ECE) Inhibitors

SLV306 (Solvay)

### NEP Inhibitors

Ecadotril (Bioproject)

### Aldosterone Antagonists

Eplerenone (Pharmacia)

### Renin Inhibitors

Aliskiren (Novartis)

SPP 500 (Roche/Speedel)

SPP600 (Speedel)

SPP 800 (Locus/Speedel)

### Angiotensin Vaccines

PMD-3117 (Protherics)

### ACE/NEP Inhibitors

AVE-7688 (Aventis)

GW-660511 (Zambon SpA)

### Na+/K+ ATPase Modulators

PST-2238 (Prassis-Sigma-Tau)

### Endothelin Antagonists

PD-156707 (Pfizer)

### Vasodilators

NCX-4016 (NicOx)

LP-805 (Pola/Wyeth)

### Naturetic Peptides

BDNP (Mayo Foundation)

### Angiotensin Receptor Blockers (ARBs)

pratosartan (Pratosartan/Boryung)

### AGE Crosslink Breakers

alagebrium chloride (Alteon)

### Endothelin Receptor Antagonists

Tezosentan (Genentech)

Ambrisentan (Myogen)

BMS193884 (BMS)

Sitaxsentan (Encysive Pharmaceuticals)

SPP301 (Roche/Speedel)

Darusentan (Myogen/Abbott)

J104132 (Banyu/Merck & Co.)

TBC3711 (Encysive Pharmaceuticals)

SB 234551 (GSK/Shionogi)

### Other Anti-Hypertension Drugs

MC4232 (University of Manitoba/Medicure)

Other Drugs from the PDR:

### Indication=Hypertension

### Accupril Tablets (Parke-Davis)

Quinapril Hydrochloride

### Accuretic Tablets (Parke-Davis)

Hydrochlorothiazide, Quinapril Hydrochloride

### Aceon Tablets (2 mg, 4 mg, 8 mg) (Solvay)

Perindopril Erbumine

### Adalat CC Tablets (Bayer)

Nifedipine

### Aldoclor Tablets (Merck)

Chlorothiazide, Methyldopa

### Aldoril Tablets (Merck)

Hydrochlorothiazide, Methyldopa

### Altace Capsules (King)

Ramipril

### Atacand Tablets (AstraZeneca LP)

Candesartan Cilexetil

### Atacand HCT 16-12.5 Tablets (AstraZeneca LP)

Candesartan Cilexetil, Hydrochlorothiazide

### Atacand HCT 32-12.5 Tablets (AstraZeneca LP)

Candesartan Cilexetil, Hydrochlorothiazide

### Avalide Tablets (Bristol-Myers Squibb)

Hydrochlorothiazide, Irbesartan

### Avapro Tablets (Bristol-Myers Squibb)

Irbesartan

### Avapro Tablets (Sanofi-Aventis)

Irbesartan

### Benicar Tablets (Sankyo)

Olmesartan Medoxomil

### Benicar HCT Tablets (Sankyo)

Hydrochlorothiazide, Olmesartan Medoxomil

### Blocadren Tablets (Merck)

Timolol Maleate

### Caduet Tablets (Pfizer)

Amlodipine Besylate, Atorvastatin Calcium

### Captopril Tablets (Mylan)

Captopril

### Cardene I.V. (ESP Pharma)

Nicardipine Hydrochloride

### Cardizem LA Extended Release Tablets (Biovail)

Diltiazem Hydrochloride

### Catapres Tablets (Boehringer Ingelheim)

Clonidine Hydrochloride

### Catapres-TTS (Boehringer Ingelheim)

Clonidine

### Clorpres Tablets (Mylan Bertek)

Chlorthalidone, Clonidine Hydrochloride

### Coreg Tablets (GlaxoSmithKline)

Carvedilol

### Corzide 40/5 Tablets (King)

Bendroflumethiazide, Nadolol

### Corzide 8015 Tablets (King)

Bendroflumethiazide, Nadolol

### Covera-HS Tablets (Searle)

Verapamil Hydrochloride

### Cozaar Tablets (Merck)

Losartan Potassium

### Demadex Tablets and Injection (Roche Laboratories)

Torsemide

### Diovan HCT Tablets (Novartis)

Hydrochlorothiazide, Valsartan

### Diovan Tablets (Novartis)

Valsartan

### Diuril Oral Suspension (Merck)

Chlorothiazide

### Diuril Tablets (Merck)

Chlorothiazide

### Dyazide Capsules (GlaxoSmithKline)

Hydrochlorothiazide, Triamterene

### DynaCirc CR Tablets (Reliant)

Isradipine

### Furosemide Tablets (Mylan)

Furosemide

### HydroDIURIL Tablets (Merck)

Hydrochlorothiazide

### Hytrin Capsules (Abbott)

Terazosin Hydrochloride

### Hyzaar 50-12.5 Tablets (Merck)

Hydrochlorothiazide, Losartan Potassium

### Hyzaar 100-25 Tablets (Merck)

Hydrochlorothiazide, Losartan Potassium

### Indapamide Tablets (Mylan)

Indapamide

### Inderal LA Long-Acting Capsules (Wyeth)

Propranolol Hydrochloride

### InnoPran XL Capsules (Reliant)

Propranolol Hydrochloride

### Inspra Tablets (Pfizer)

Eplerenone

### Inversine Tablets (Targacept)

Mecamylamine Hydrochloride

### Isoptin SR Tablets (Abbott)

Verapamil Hydrochloride

### Lotensin Tablets (Novartis)

Benazepril Hydrochloride

### Lotensin HCT Tablets (Novartis)

Benazepril Hydrochloride, Hydrochlorothiazide

### Lotrel Capsules (Novartis)

Amlodipine Besylate, Benazepril Hydrochloride

### Mavik Tablets (Abbott)

Trandolapril

### Maxzide Tablets (Mylan Bertek)

Hydrochlorothiazide, Triamterene

### Maxzide-25 mg Tablets (Mylan Bertek)

Hydrochlorothiazide, Triamterene

### Micardis Tablets (Boehringer Ingelheim)

Telmisartan

### Micardis HCT Tablets (Boehringer Ingelheim)

Hydrochlorothiazide, Telmisartan

### Midamor Tablets (Merck)

Amiloride Hydrochloride

### Moduretic Tablets (Merck)

Amiloride Hydrochloride, Hydrochlorothiazide

### Nadolol Tablets (Mylan)

Nadolol

### Norvasc Tablets (Pfizer)

Amlodipine Besylate

### Prinivil Tablets (Merck)

Lisinopril

### Prinzide Tablets (Merck)

Hydrochlorothiazide, Lisinopril

### Sular Tablets (First Horizon)

Nisoldipine

### Tarka Tablets (Abbott)

Trandolapril, Verapamil Hydrochloride

### Teveten Tablets (Biovail)

Eprosartan Mesylate

### Teveten HCT Tablets (Biovail)

Eprosartan Mesylate, Hydrochlorothiazide

### Tiazac Capsules (Forest)

Diltiazem Hydrochloride

### Timolide Tablets (Merck)

Hydrochlorothiazide, Timolol Maleate

### Toprol-XL Tablets (AstraZeneca LP)

Metoprolol Succinate

### Uniretic Tablets (Schwarz)

Hydrochlorothiazide, Moexipril Hydrochloride

### Univasc Tablets (Schwarz)

Moexipril Hydrochloride

### Vaseretic Tablets (Merck)

Enalapril Maleate, Hydrochlorothiazide

### Vasotec I.V. Injection (Merck)

Enalapril Maleate

### Verelan PM Capsules (Schwarz)

Verapamil Hydrochloride

### Zaroxolyn Tablets (Celltech)

Metolazone

1. A method of treating or preventing a condition selected from the group consisting of obesity, insulin resistance, hyperglycemia, and type 2 diabetes, the method comprising:
administering to an animal an agent known to reduce ER stress.

2. The method of claim 1 further comprising administering an agent selected from the group consisting of anti-diabetic agents, anti-obesity agents, anti-dyslipidemia agents, anti-atherosclerosis agents, and anti-hypertensive agents.

3. The method of claim 2, wherein the anti-diabetic agent is selected from the group consisting of biguanides, metformin, sulfonylureas, insulin, analogs of insulin, PPARg agonists, meglitinides, and DPP-IV inhibitors.

4. The method of claim 2, wherein the anti-obesity agent is selected from the group consisting of pancreatic lipase inhibitors, serotonin reuptake inhibitors, norepinephrine reuptake inhibitors, noradrenergic anorectic agents, peripherally acting agents, centrally acting agents, and thermogenic agents.

5. The method of claim 2, wherein the anti-dyslipidemia agent or anti-atherosclerosis agent is selected from the group consisting of HMG-CoA reductase inhibitors, niacin, anti-platelets, ACE inhibitors, aspirin, analogs of aspirin, and MCP-1 inhibitors.

6. The method of claim 2, wherein the anti-hypertensive agent is selected from the group consisting of diuretics, beta-blockers, Ca⁺² channel blockers, ACE inhibitors, and AT-II inhibitors.

7. (canceled)

8. (canceled)

9. (canceled)

10. The method of claim 1, wherein the animal is a mammal.

11. The method of claim 1, wherein the animal is a human.

12. The method of claim 1, wherein the step of administering comprises administering an agent orally, administering an agent parenterally or administering an agent intravenously.

13. (canceled)

14. (canceled)

15. The method of claim 1, wherein the agent is a chemical chaperone selected from the group consisting of glycerol, D₂O, dimethylsulfoxide (DMSO), glycine betaine (betaine), glycerolphosphocholine (GPC), methylamines, and trimethylamine N-oxide (TMAO.

16-19. (canceled)

20. The method of claim 1, wherein the agent is a derivative or salt of trimethylamine N-oxide (TMAO).

21. The method of claim 1, wherein the agent is of the formula: 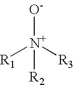
wherein
R₁, R₂, and R₃ are independently hydrogen, halogen, or lower C₁-C₆ alkyl; or a pharmaceutically-acceptable salt thereof; or a mixture thereof.

22. (canceled)

23. The method of claim 1, wherein the agent is phenyl butyric acid (PBA) or a derivative, salt, or isomer of PBA.

24. (canceled)

25. The method of claim 1, wherein the agent is of the formula: 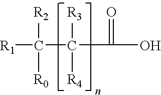
wherein n is 1 or 2;
R₀ is aryl, heteroaryl, or phenoxy, the aryl and phenoxy being unsubstituted or substituted with, independently, one or more halogen, hydroxy or lower alkyl;
R₁ and R₂ are independently H, lower alkoxy, hydroxy, lower alkyl or halogen; and
R₃ and R₄ are independently H, lower alkyl, lower alkoxy or halogen; or a pharmaceutically-acceptable derivative or salt thereof.

26-33. (canceled)

34. The method of claim 1, wherein the agent is tauroursodeoxycholic acid (TUDCA) or a derivative, salt, or isomer of TUDCA.

35. (canceled)

36. The method of claim 1, wherein the agent is of the formula: 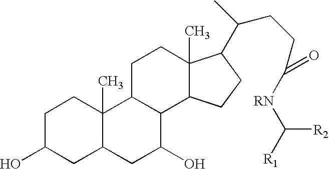
wherein R is —H or C₁-C₄ alkyl;
R₁ is —CH₂—SO₃R₃ and R₂ is —H; or R₁ is —COOH and R₂ is —CH₂—CH₂—CONH₂,
—CH₂—CONH₂, —CH₂—CH₂—SCH₃ or —CH₂—S—CH₂—COOH; and
R3 is —H or the residue of a basic amino acid, or
a pharmaceutically acceptable salt or derivative thereof.

37. (canceled)

38. (canceled)

39. The method of claim 1, wherein the agent is administered at a dose ranging from 100 mg/kg/day to 5 g/kg/day, a dose ranging from 500 mg/kg/day to 3 g/kg/day or a dose ranging from 500 mg/kg/day to 1 g/kg/day.

40. (canceled)

41. (canceled)

42. A method of treating or preventing obesity, the method comprising administering to a human an agent known to reduce ER stress.

43. A method of: reducing blood glucose levels; or increasing insulin sensitivity, the method comprising administering to an animal an agent selected from the group consisting of PBA, TUDCA, and derivatives thereof.

44. (canceled)

45. A method of screening for agents to: treat or prevent obesity, insulin resistance, or diabetes; modulate insulin action or insulin receptor signaling; or reduce ER stress, the method comprising steps of:
providing an agent to be screened;
contacting the agent with a cell; and
determining whether ER stress markers are reduced.

46. (canceled)

47. The method of claim 45, wherein ER stress markers are selected from the group consisting of spliced forms of XBP-1, phosphorylation status of PERK, phosphorylation of eIF2a, mRNA levels of GRP78/BIP, protein levels of GRP78/BIP, and JNK activity.

48. The method of claim 45, wherein the cell is a mammalian cell, a human cell, an adipocyte or a hepatocyte.

49-51. (canceled)

52. The method of claim 45, wherein the cell is experiencing ER stress or has been treated with tunicamycin or thapsigargin to induce ER stress.

53. (canceled)

54. (canceled)

55. A method of screening for agents that prevent ER stress, the method comprising steps of:
providing an agent to be screened;
contacting the agent with a cell;
subsequently contacting the cell contacted with the agent with an ER stress inducer; and determining whether ER stress markers are reduced.

56. The method of claim 55, wherein the ER stress inducer is selected from the group consisting of tunicamycin and thapsigargin.

57. (canceled)

58. (canceled)

59. A pharmaceutical composition comprising (1) an agent known to reduce ER stress, and (2) an agent selected from the group consisting of anti-diabetic agents, anti-obesity agents, antidyslipidemia agents, anti-atherosclerosis agents, and anti-hypertensive agents.

60-64. (canceled)

65. A method of reducing ER stress or treating or preventing a disease associated with ER stress, the method comprising steps of:
administering to an animal an agent selected from the group consisting of PBA, TUDCA, and derivatives thereof.

66. A method of diagnosing insulin resistance, hyperglycemia, or type 2 diabetes, the method comprising:
measuring the level of expression of at least one ER stress marker, wherein an increase in the level of the ER stress marker indicates that the subject is at risk of insulin resistance, hyperglycemia, or type 2 diabetes.

67. The method of claim 66, wherein the ER stress marker is selected from the group consisting of spliced forms of XBP-1, phosphorylation status of PERK, phosphorylation of eIF2a, mRNA levels of GRP78/BIP, protein levels of GRP78/BIP, and JNK activity.

68. (canceled)

69. A method of modulating PERK, IRE-1a. JNK, IRS-1, IRS-2, Akt, or insulin receptor activity comprising:
administering to an animal an agent selected from the group consisting of PBA, TUDCA, and derivatives thereof

70-76. (canceled)

77. A method of increasing insulin action or insulin receptor signaling, the method comprising:
administering to an animal an agent selected from the group consisting of PBA, TUDCA, and derivatives thereof.

78. (canceled)

79. A method of modulating insulin receptor signaling comprising:
administering to an animal an agent known to reduce ER stress.

80-82. (canceled)
